(19) United States
(12) Patent Application Publication
(10) Pub. No.: US 2026/0130976 A1
(43) Pub. Date: May 14, 2026

(54) NOVEL TUMOR-SPECIFIC ANTIGENS FOR MYELOID LEUKEMIA AND USES THEREOF

(71) Applicant: UNIVERSITÉ DE MONTRÉAL, Montréal, Québec (CA)

(72) Inventors: Claude PERREAULT, Montréal, Québec (CA); Pierre THIBAULT, Montreal, Québec (CA); Marie-Pierre HARDY, Terrebonne, Québec (CA)

(21) Appl. No.: 19/119,677

(22) PCT Filed: Oct. 6, 2023
(86) PCT No.: PCT/CA2023/051329

§ 371 Date: Apr. 9, 2025

Related U.S. Application Data
(60) Provisional application No. 63/379,025, filed on Oct. 11, 2022.

(51) Int. Cl. A61K 39/00

(52) U.S. Cl. CPC A61K 39/001102; A61K 2039/53; A61K 2039/55588

(57) ABSTRACT

Acute myeloid leukemia (AML) has not benefited from innovative immunotherapies, mainly because of the lack of actionable immune targets. Novel tumor-specific antigens (TSAs) shared by a large proportion of AML cells are described herein. Most of the TSAs described herein derives from aberrantly expressed unmutated genomic sequences, such as intronic and intergenic sequences, which are not expressed in normal tissues. Nucleic acids, compositions, cells and vaccines derived from these TSAs are described. The use of the TSAs, nucleic acids, compositions, cells and vaccines for the treatment of myelodysplastic syndrome (MDS) or leukemia such as AML is also described.

### CROSS REFERENCE TO RELATED APPLICATIONS

The present application claims the benefit of U.S. provisional patent application No. 63/379,025 filed Oct. 11, 2022, which is incorporated herein by reference in its entirety.

### SEQUENCE LISTING

A sequence listing is submitted herewith as an XML file named 17971-00070-AD.xml, that was created on Oct. 2, 2023, and having a size of −20 kilobytes. The content of the aforementioned file is hereby incorporated by reference in its entirety.

### TECHNICAL FIELD

The present invention generally relates to the field of cancer, and more particularly to the treatment of cancer such as myeloid leukemia.

### BACKGROUND ART

Acute myeloid leukemia (AML), the most aggressive hematologic malignancy, is a heterogeneous disease characterized by aberrant epigenetic patterning, disturbed mitochondrial proteostasis and a relatively low number of mutations (Li et al., 2016; Ntziachristos et al., 2016; Ishizawa et al., 2019; Fennell et al., 2019). Notably, genetic and epigenetic changes in AML may precede diagnosis by many years (Abelson et al., 2018; Desai et al., 2018). Also, cure requires not only elimination of bulk tumor cells but also of leukemic stem cells (Shlush et al., 2017; Boyd et al., 2018). Currently, most patients relapse following chemotherapy, with 5-year overall survival of 40% for patients <60 years and only 10-20% for those aged >60 years (who represent the majority of AML cases) (Vasu et al., 2018).

Over the last few years, enthusiasm for cancer immunotherapy has been fueled mainly by two major breakthroughs: i) immune checkpoint therapy for treatment melanoma and selected types of solid tumors and ii) chimeric antigen receptors for treatment of lymphoid malignancies. However, AML has not benefited from such innovations, mainly because of the lack of actionable immune targets. In accordance with the notion that major histocompatibility complex MHC-associated peptides (MAPs) recognized by T cells are at the core of anti-cancer responses (Coulie et al., 2014), evidence suggests that AML cells should present immunogenic MAPs to CD8 T cells: i) AML cells express a high density of MHC class I molecules (Berlin et al., 2015) and ii) the bone marrow of AML patients contains CD8 T cells with phenotypic and transcriptional features of exhaustion (and therefore of antigen recognition) (Knaus et al., 2018). However, the nature of AML antigens able to elicit protective immune responses remains elusive.

The first class of MAPs that attracted the attention of cancer immunologists are tumor-associated antigens (TAAs) that are overexpressed in tumor cells relative to normal cells. Since high-affinity T cells recognizing self-antigens are eliminated by the central tolerance process of thymic selection, TAAs are essentially recognized by low affinity T cells. Accordingly, TAA-based vaccines have had no convincing impact on AML evolution. Disappointing results were notably obtained with the most studied AML TAA: Wilms' Tumor 1 (WT1) (Di Stasi et al., 2015; Maslak et al., 2018; Rashidi and Walter, 2016). Importantly, a recent report showed that TCR gene therapy (in which T cells are engineered to express a high affinity TCR against a selected antigen) targeted against a WT1-derived peptide could durably prevent relapse in recipients of allogeneic hematopoietic stem cell transplantation (Chapuis et al., 2019). Overall, these studies suggest that WT1-derived peptides are poorly immunogenic and need to be targeted with engineered T cells to reach their full therapeutic potential.

In contrast to TAAs, tumor specific antigens (TSAs) are MAPs solely presented by tumor cells. So far, mutated TSAs (mTSAs), also known as neoantigens, have attracted substantial attention lately in quests for vaccines against solid tumors. Indeed, mTSAs can be highly immunogenic because they are not found in medullary thymic cells (mTECs) which induce central tolerance. However, mTSAs present two caveats. First, they are generally unique to each patients' tumors (private neoantigens). Second, they are less common than initially predicted (Knaus et al., 2018). Consistent with the low mutational burden of AML cells, only one mTSA has been validated by mass spectrometry (MS) analyses of primary AML cells (van der Lee et al., 2019). The therapeutic potential of this mTSA deriving from a frameshift in the NPM1 gene has yet to be evaluated, but according to available evidence, it does not elicit spontaneous immune responses in AML patients (van der Lee et al., 2019).

In view of this, there is a pressing need to identify the antigens that can elicit therapeutic immune responses again AML. Such antigens could be used as vaccines (±immune checkpoint inhibitors) or as targets for T-cell receptor-based approaches (cell therapy, bispecific biologics).

The present description refers to a number of documents, the content of which is herein incorporated by reference in their entirety.

### SUMMARY

In various aspects and embodiments, the present disclosure provides the following items 1 to 59:
1. A tumor antigen peptide (TAP) comprising or consisting of one of the following amino acid sequences:

| Peptide sequence | SEQ ID NO: |
|---|---|
| SIFSEILAV | 1 |
| SPFVRTQEF | 2 |
| AAPGRAVGGM | 3 |
| RTWKSVLSK | 4 |
| SLSSLVVLV | 5 |
| RTPGLIRVGK | 6 |
| STWRLHLR | 7 |
| EFWIRENQR | 8 |
| APGSARAMI | 9 |
| EIRVDFLEK | 10 |
| KAFLTTAQK | 11 |

or a nucleic acid encoding said TAP.
2. The TAP or nucleic acid of item 1, wherein the TAP binds to an HLA-A*02:01 molecule and comprises or consists of the sequence of SEQ ID NO: 1 or 5.
3. The TAP or nucleic acid of item 1, wherein the TAP binds to an HLA-A*03:01 molecule and comprises or consists of the sequence of SEQ ID NO: 4, 6 or 11.
4. The TAP or nucleic acid of item 1, wherein the TAP binds to an HLA-A*26:01 molecule and comprises or consists of the sequence of SEQ ID NO: 1 or 7.
5. The TAP or nucleic acid of item 1, wherein the TAP binds to an HLA-A*30:01 molecule and comprises or consists of the sequence of SEQ ID NO: 6.
6. The TAP or nucleic acid of item 1, wherein the TAP binds to an HLA-A*33:01 molecule and comprises or consists of the sequence of SEQ ID NO: 7.
7. The TAP or nucleic acid of item 1, wherein the TAP binds to an HLA-A*33:03 molecule and comprises or consists of the sequence of SEQ ID NO: 8.
8. The TAP or nucleic acid of item 1, wherein the TAP binds to an HLA-A*34:02 molecule and comprises or consists of the sequence of SEQ ID NO: 10 or 11.
9. The TAP or nucleic acid of item 1, wherein the TAP binds to an HLA-B*07:02 molecule and comprises or consists of the sequence of SEQ ID NO: 2, 3 or 9.
10. The TAP or nucleic acid of item 1, wherein the TAP binds to an HLA-B*08:01 molecule and comprises or consists of the sequence of SEQ ID NO: 2.
11. The TAP or nucleic acid of item 1, wherein the TAP binds to an HLA-B*27:05 molecule and comprises or consists of the sequence of SEQ ID NO: 4.
12. The TAP or nucleic acid of item 1, wherein the TAP binds to an HLA-B*39:01 molecule and comprises or consists of the sequence of SEQ ID NO: 2.
13. The TAP or nucleic acid of any one of items 1-12, which is encoded by a sequence located a non-protein coding region of the genome.
14. The TAP or nucleic acid of item 13, wherein said non-protein coding region of the genome is an intergenic region.
15. The TAP or nucleic acid of item 13, wherein said non-protein coding region of the genome is a long non-coding RNAs.
16. The TAP or nucleic acid of item 13, wherein said non-protein coding region of the genome is an intron.
17. A combination comprising at least two of the TAPs or nucleic acids defined in any one of items 1-16.
18. A synthetic long peptide (SLP) comprising at least one of the amino acid sequences defined in item 1.
19. The TAP, combination, SLP or nucleic acid of any one of items 1 to 18, wherein the nucleic acid is an mRNA.
20. The TAP, combination, SLP or nucleic acid of item 19, wherein the mRNA comprises one or more 5′-end modifications, 3′-end modifications, and/or modified nucleosides to increase stability, improve translation and/or reduce immunogenicity of the mRNA.
21. The TAP, combination, SLP or nucleic acid of any one of items 1 to 18, wherein the nucleic acid is a DNA.
22. The TAP, combination, SLP, or nucleic acid of any one of items 1 to 21, wherein the nucleic acid is a component of a viral vector.
23. A vesicle or particle comprising the TAP, nucleic acid, combination or SLP of any one of items 1 to 22.
24. The vesicle or particle of item 23, wherein the vesicle is a lipid nanoparticle (LNP).
25. The vesicle or particle of item 23 or 24, which comprises a cationic lipid.
26. A composition comprising the TAP, nucleic acid, combination or SLP of any one of items 1 to 22, or the vesicle or particle of any one of items 23-25, and a pharmaceutically acceptable carrier.
27. A vaccine comprising the TAP, nucleic acid, combination or SLP of any one of items 1 to 22, the vesicle or particle of any one of items 23-25, or the composition of item 26, and an adjuvant.
28. An isolated major histocompatibility complex (MHC) class I molecule comprising the TAP of any one of items 1-16 in its peptide binding groove.
29. The isolated MHC class I molecule of item 28, which is in the form of a multimer.
30. The isolated MHC class I molecule of item 29, wherein said multimer is a tetramer.
31. An isolated cell comprising (i) the TAP of any one of items 1-16, (ii) the combination of item 17; (iii) the SLP of item 18; or (iv) a vector comprising a nucleotide sequence encoding the TAP of any one of items 1-16, the combination of item 17 or the SLP of item 18.
32. An isolated cell expressing at its surface major histocompatibility complex (MHC) class I molecules comprising the TAP or combination of any one of items 1-17 in their peptide binding groove.
33. The cell of item 31 or 32, which is an antigen-presenting cell (APC).
34. The cell of item 33, wherein said APC is a dendritic cell.
35. A T-cell receptor (TCR) that specifically recognizes the isolated MHC class I molecule of any one of items 28-30 and/or MHC class I molecules expressed at the surface of the cell of any one of items 32-34.
36. The TCR of item 35, which is a soluble TCR.
37. An antibody or an antigen-binding fragment thereof that specifically binds to the isolated MHC class I molecule of any one of items 28-30 and/or MHC class I molecules expressed at the surface of the cell of any one of items 32-34.
38. The TCR of item 35 or 36, or the antibody or antigen-binding fragment thereof according to item 37, which is a bispecific TCR or a bispecific antibody or antigen-binding fragment thereof.
39. The TCR, antibody or antigen-binding fragment thereof according to item 38, wherein the bispecific antibody or antigen-binding fragment thereof is a single-chain diabody (scDb).
40. The TCR, antibody or antigen-binding fragment thereof according to item 38 or 39, wherein the bispecific TCR, antibody or antigen-binding fragment thereof also specifically binds to a T cell signaling molecule.
41. The TCR, antibody or antigen-binding fragment thereof according to item 40, wherein the T cell signaling molecule is a CD3 chain.
42. An isolated cell expressing at its cell surface the TCR of item 35.
43. The isolated cell of item 42, which is a CD8⁺ T lymphocyte.
44. A cell population comprising at least 0.5% of the isolated cell as defined in item 42 or 43.
45. A method of treating myelodysplastic syndrome (MDS) or leukemia in a subject comprising administering to the subject an effective amount of:
   (a) a TAP comprising or consisting of any one of the sequences set forth in SEQ ID NOs: 1-11 or any combination thereof, or a synthetic long peptide (SLP) comprising at least one of the sequences set forth in SEQ ID NOs: 1-11;
   (b) at least one nucleic acid encoding the TAP, combination thereof or SLP defined in (a); (c) a vesicle or particle comprising the TAP, combination thereof or SLP defined in (a) or the at least one nucleic acid defined in (b);
   (d) a composition comprising the TAP, combination thereof or SLP defined in (a), the at least one nucleic acid defined in (b), or the vesicle or particle defined in (c), and a pharmaceutically acceptable carrier;
   (e) a vaccine comprising the TAP, combination thereof or SLP defined in (a), the at least one nucleic acid defined in (b), the vesicle or particle defined in (c), or the composition defined in (d), and an adjuvant;
   (f) a cell expressing at its surface major histocompatibility complex (MHC) class I molecules comprising the TAP or combination thereof defined in (a) in their peptide binding groove;
   (g) a cell expressing at its cell surface a T-cell receptor (TCR) that specifically recognizes MHC class I molecules expressed at the surface of the cell defined in (f); or
   (h) a soluble TCR, an antibody or an antigen-binding fragment thereof that specifically binds to the MHC class I molecules expressed at the surface of the cell defined in (f).
46. The method of item 45, wherein the leukemia is a myeloid leukemia.
47. The method of item 46, wherein said myeloid leukemia is acute myeloid leukemia (AML).
48. The method of any one of items 45 to 47, further comprising administering at least one additional antitumor agent or therapy to the subject.
49. The method of item 48, wherein said at least one additional antitumor agent or therapy is a chemotherapeutic agent, immunotherapy, an immune checkpoint inhibitor, radiotherapy or surgery.
50. Use of:
   (a) a TAP comprising or consisting of any one of the sequences set forth in SEQ ID NOs: 1-11 or any combination thereof, or a synthetic long peptide (SLP) comprising at least one of the sequences set forth in SEQ ID NOs: 1-11;
   (b) at least one nucleic acid encoding the TAP, combination thereof or SLP defined in (a);
   (c) a vesicle or particle comprising the TAP, combination thereof or SLP defined in (a) or the at least one nucleic acid defined in (b);
   (d) a composition comprising the TAP, combination thereof or SLP defined in (a), the at least one nucleic acid defined in (b), or the vesicle or particle defined in (c), and a pharmaceutically acceptable carrier;
   (e) a vaccine comprising the TAP, combination thereof or SLP defined in (a), the at least one nucleic acid defined in (b), the vesicle or particle defined in (c), or the composition defined in (d), and an adjuvant;
   (f) a cell expressing at its surface major histocompatibility complex (MHC) class I molecules comprising the TAP or combination thereof defined in (a) in their peptide binding groove;
   (g) a cell expressing at its cell surface a T-cell receptor (TCR) that specifically recognizes MHC class I molecules expressed at the surface of the cell defined in (f); or
   (h) a soluble TCR, an antibody or an antigen-binding fragment thereof that specifically binds to the MHC class I molecules expressed at the surface of the cell defined in (f); for treating myelodysplastic syndrome (MDS) or leukemia in a subject, or for the manufacture of a medicament for treating MDS or leukemia in a subject.
51. The use of item 50, wherein said leukemia is a myeloid leukemia.
52. The use of item 51, wherein said myeloid leukemia is acute myeloid leukemia (AML).
53. The use of any one of items 50 to 52, further comprising the use at least one additional antitumor agent or therapy to the subject.
54. The use of item 53, wherein said at least one additional antitumor agent or therapy is a chemotherapeutic agent, immunotherapy, an immune checkpoint inhibitor, radiotherapy or surgery.
55. An agent for use in treating myelodysplastic syndrome (MDS) or leukemia in a subject, wherein the agent is:
   (a) a TAP comprising or consisting of any one of the sequences set forth in SEQ ID NOs: 1-11 or any combination thereof, or a synthetic long peptide (SLP) comprising at least one of the sequences set forth in SEQ ID NOs: 1-11;
   (b) at least one nucleic acid encoding the TAP, combination thereof or SLP defined in (a);
   (c) a vesicle or particle comprising the TAP, combination thereof or SLP defined in (a) or the at least one nucleic acid defined in (b);
   (d) a composition comprising the TAP, combination thereof or SLP defined in (a), the at least one nucleic acid defined in (b), or the vesicle or particle defined in (c), and a pharmaceutically acceptable carrier;
   (e) a vaccine comprising the TAP, combination thereof or SLP defined in (a), the at least one nucleic acid defined in (b), the vesicle or particle defined in (c), or the composition defined in (d), and an adjuvant;
   (f) a cell expressing at its surface major histocompatibility complex (MHC) class I molecules comprising the TAP or combination thereof defined in (a) in their peptide binding groove;
   (g) a cell expressing at its cell surface a T-cell receptor (TCR) that specifically recognizes MHC class I molecules expressed at the surface of the cell defined in (f); or
   (h) a soluble TCR, an antibody or an antigen-binding fragment thereof that specifically binds to the MHC class I molecules expressed at the surface of the cell defined in (f).
56. The agent for use of item 55, wherein the leukemia is a myeloid leukemia.
57. The agent for use of item 56, wherein the myeloid leukemia is acute myeloid leukemia (AML).
58. The agent for use of any one of items 55 to 57, further comprising the use at least one additional antitumor agent or therapy to the subject.
59. The agent for use of item 58, wherein said at least one additional antitumor agent or therapy is a chemotherapeutic agent, immunotherapy, an immune checkpoint inhibitor, radiotherapy or surgery.

Other objects, advantages and features of the present disclosure will become more apparent upon reading of the following non-restrictive description of specific embodiments thereof, given by way of example only with reference to the accompanying drawings.

### DETAILED DISCLOSURE

The use of the terms “a” and “an” and “the” and similar referents in the context of describing the technology (especially in the context of the following claims) are to be construed to cover both the singular and the plural, unless otherwise indicated herein or clearly contradicted by context.

The terms “comprising”, “having”, “including”, and “containing” are to be construed as open-ended terms (i.e., meaning “including, but not limited to”) unless otherwise noted.

All methods described herein can be performed in any suitable order unless otherwise indicated herein or otherwise clearly contradicted by context.

The use of any and all examples, or exemplary language (“e.g.”, “such as”) provided herein, is intended merely to better illustrate embodiments of the claimed technology and does not pose a limitation on the scope unless otherwise claimed.

No language in the specification should be construed as indicating any non-claimed element as essential to the practice of embodiments of the claimed technology.

Herein, the term “about” has its ordinary meaning. The term “about” is used to indicate that a value includes an inherent variation of error for the device or the method being employed to determine the value, or encompass values close to the recited values, for example within 10% of the recited values (or range of values).

Recitation of ranges of values herein are merely intended to serve as a shorthand method of referring individually to each separate value falling within the range, unless otherwise indicated herein, and each separate value is incorporated into the specification as if it were individually recited herein. All subsets of values within the ranges are also incorporated into the specification as if they were individually recited herein.

Where features or aspects of the disclosure are described in terms of Markush groups or list of alternatives, those skilled in the art will recognize that the disclosure is also thereby described in terms of any individual member, or subgroup of members, of the Markush group or list of alternatives.

Unless specifically defined otherwise, all technical and scientific terms used herein shall be taken to have the same meaning as commonly understood by one of ordinary skill in the art (e.g., in stem cell biology, cell culture, molecular genetics, immunology, immunohistochemistry, protein chemistry, and biochemistry).

Unless otherwise indicated, the recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

In the studies described herein, the present inventors have identified TSA candidates from AML specimens using a proteogenomic-based approach. The novel TSA candidates identified herein may be useful, e.g., for immunotherapies and vaccines against cancers expressing the TSA candidates, such as myeloid leukemias.

The present disclosure relates to a tumor antigen peptide (TAP) (or tumor-specific peptide), such as a leukemia TAP, comprising, or consisting of, one of the following amino acid sequences:

| Peptide sequence | SEQ ID NO: |
|---|---|
| SIFSEILAV | 1 |
| SPFVRTQEF | 2 |
| AAPGRAVGGM | 3 |
| RTWKSVLSK | 4 |
| SLSSLVVLV | 5 |
| RTPGLIRVGK | 6 |
| STWRLHLR | 7 |
| EFWIRENQR | 8 |
| APGSARAMI | 9 |
| EIRVDFLEK | 10 |
| KAFLTTAQK | 11 |

In general, peptides such as tumor antigen peptides (TAPs) presented in the context of HLA class I vary in length from about 7 or 8 to about 15, or preferably 8 to 14 amino acid residues. In some embodiments of the methods of the disclosure, longer peptides comprising the TAP sequences defined herein are artificially loaded into cells such as antigen presenting cells (APCs), processed by the cells and the TAP is presented by MHC class I molecules at the surface of the APC. In this method, peptides/polypeptides longer than 15 amino acid residues can be loaded into APCs, are processed by proteases in the APC cytosol providing the corresponding TAP as defined herein for presentation. In some embodiments, the precursor peptide/polypeptide that is used to generate the TAP defined herein is for example 1000, 500, 400, 300, 200, 150, 100, 75, 50, 45, 40, 35, 30, 25, 20 or 15 amino acids or less. Thus, all the methods and processes using the TAPs described herein include the use of longer peptides or polypeptides (including the native protein), i.e., tumor antigen precursor peptides/polypeptides, to induce the presentation of the “final” 8-14 TAP following processing by the cell (APCs). In some embodiments, the herein-mentioned TAP is about 8 to 14, 8 to 13, or 8 to 12 amino acids long (e.g., 8, 9, 10, 11, 12 or 13 amino acids long), small enough for a direct fit in an HLA class I molecule. In an embodiment, the TAP comprises 20 amino acids or less, preferably 15 amino acids or less, more preferably 14 amino acids or less. In an embodiment, the TAP comprises at least 7 amino acids, preferably at least 8 amino acids, more preferably at least 9 amino acids.

The term “amino acid” as used herein includes both L- and D-isomers of the naturally occurring amino acids as well as other amino acids (e.g., naturally-occurring amino acids, non-naturally-occurring amino acids, amino acids which are not encoded by nucleic acid sequences, etc.) used in peptide chemistry to prepare synthetic analogs of TAPs. Examples of naturally occurring amino acids are glycine, alanine, valine, leucine, isoleucine, serine, threonine, etc. Other amino acids include for example non-genetically encoded forms of amino acids, amino acid analogs as well as a conservative substitution of an L-amino acid. Naturally-occurring non-genetically encoded amino acids and amino acid analogs include, for example, beta-alanine, 3-amino-propionic acid, 2,3-diaminopropionic acid, alpha-aminoisobutyric acid (Aib), 4-amino-butyric acid, N-methylglycine (sarcosine), hydroxyproline, ornithine (e.g., L-ornithine), citrulline, t-butylalanine, t-butylglycine, N-methylisoleucine, phenylglycine, cyclohexylalanine, norleucine (Nle), norvaline, 2-napthylalanine, pyridylalanine, 3-benzothienyl alanine, 4-chlorophenylalanine, 2-fluorophenylalanine, 3-fluorophenylalanine, 4-fluorophenylalanine, penicillamine, 1,2,3,4-tetrahydro-isoquinoline-3-carboxylix acid, beta-2-thienylalanine, methionine sulfoxide, L-homoarginine (Hoarg), N-acetyl lysine, 2-amino butyric acid, 2-amino butyric acid, 2,4,-diaminobutyric acid (D- or L-), p-aminophenylalanine, N-methylvaline, homocysteine, homoserine (HoSer), cysteic acid, epsilon-amino hexanoic acid, delta-amino valeric acid, benzyloxy-tyrosine, p-phenylalanine or 2,3-diaminobutyric acid (D- or L-), etc. These amino acids are well known in the art of biochemistry/peptide chemistry. Thus, one or more of the amino acids in the TAPs described herein (SEQ ID NO:1-11) may be replaced by a non-genetically encoded amino acid and/or an amino acid analog. The TAPs may also be modified to improve the proteolytic stability of the peptides, for example by the incorporation of methyl-amino acids, p-amino acids or peptoids. In an embodiment, the TAP comprises only naturally-occurring amino acids.

In embodiments, the TAPs described herein include peptides with altered sequences containing substitutions of functionally equivalent amino acid residues, relative to the herein-mentioned sequences. For example, one or more amino acid residues within the sequence can be substituted by another amino acid of a similar polarity (having similar physico-chemical properties) which acts as a functional equivalent, resulting in a silent alteration. Substitution for an amino acid within the sequence may be selected from other members of the class to which the amino acid belongs. For example, positively charged (basic) amino acids include arginine, lysine and histidine (as well as homoarginine and ornithine). Nonpolar (hydrophobic) amino acids include leucine, isoleucine, alanine, phenylalanine, valine, proline, tryptophan and methionine. Uncharged polar amino acids include serine, threonine, cysteine, tyrosine, asparagine and glutamine. Negatively charged (acidic) amino acids include glutamic acid and aspartic acid. The amino acid glycine may be included in either the nonpolar amino acid family or the uncharged (neutral) polar amino acid family. Substitutions made within a family of amino acids are generally understood to be conservative substitutions. The herein-mentioned TAP may comprise all L-amino acids, all D-amino acids or a mixture of L- and D-amino acids. In an embodiment, the herein-mentioned TAP comprises all L-amino acids.

In an embodiment, in the sequences of the TAPs comprising or consisting of one of sequences of SEQ ID NOs: 1-11, the amino acid residues that do not substantially contribute to interactions with the T-cell receptor may be modified by replacement with other amino acid whose incorporation does not substantially affect T-cell reactivity and does not eliminate binding to the relevant MHC.

The TAP may also be modified by replacing one or more of the amide bonds (or peptide bonds) of the peptide that may improve chemical stability and/or enhanced biological/pharmacological properties (e.g., half-life, absorption, potency, efficiency, etc.). Typical peptide bond replacements include esters, polyamines and derivatives thereof as well as substituted alkanes and alkenes, such as aminomethyl and ketomethylene. For example, the above-mentioned TAP may have one or more amide bonds replaced by linkages such as —CH₂NH—, —CH₂S—, —CH₂—CH₂—, —CH═CH— (cis or trans), —CH₂SO—, —CH(OH)CH₂—, or —COCH₂.

The TAP may also be N- and/or C-terminally capped or modified to prevent degradation, increase stability, affinity and/or uptake. Thus, in another aspect, the present disclosure provides a modified TAP of the formula Z¹—X—Z², wherein X is a TAP comprising, or consisting of, one of the amino acid sequences of SEQ ID NOs: 1-11.

In an embodiment, the amino terminal residue (i.e., the free amino group at the N-terminal end) of the TAP is modified (e.g., for protection against degradation), for example by covalent attachment of a moiety/chemical group (Z¹). Z¹ may be a straight chained or branched alkyl group of one to eight carbons, or an acyl group (R—CO—), wherein R is a hydrophobic moiety (e.g., acetyl, propionyl, butanyl, iso-propionyl, or iso-butanyl), or an aroyl group (Ar—CO—), wherein Ar is an aryl group. In an embodiment, the acyl group is a C₁-C₁₆ or C₃-C₁₆ acyl group (linear or branched, saturated or unsaturated), in a further embodiment, a saturated C₁-C₆ acyl group (linear or branched) or an unsaturated C₃-C₆ acyl group (linear or branched), for example an acetyl group (CH₃—CO—, Ac). In an embodiment, Z¹ is absent. The carboxy terminal residue (i.e., the free carboxy group at the C-terminal end of the TAP) of the TAP may be modified (e.g., for protection against degradation), for example by amidation (replacement of the OH group by a NH₂ group), thus in such a case Z² is a NH₂ group. In an embodiment, Z² may be an hydroxamate group, a nitrile group, an amide (primary, secondary or tertiary) group, an aliphatic amine of one to ten carbons such as methyl amine, iso-butylamine, iso-valerylamine or cyclohexylamine, an aromatic or arylalkyl amine such as aniline, napthylamine, benzylamine, cinnamylamine, or phenylethylamine, an alcohol or CH₂OH. In an embodiment, Z² is absent. In an embodiment, the TAP comprises one of the amino acid sequences of SEQ ID NOs: 1-11. In an embodiment, the TAP consists of one of the amino acid sequences of SEQ ID NOs: 1-11, i.e., wherein Z¹ and Z² are absent.

In another aspect, the present disclosure provides a TAP binding to an HLA-A*02:01 molecule, comprising or consisting of the sequence of SEQ ID NO: 1 or 5.

In another aspect, the present disclosure provides a TAP binding to an HLA-A*03:01 molecule, comprising or consisting of the sequence of SEQ ID NO: 4, 6 or 11.

In another aspect, the present disclosure provides a TAP binding to an HLA-A*26:01 molecule, comprising or consisting of the sequence of SEQ ID NO: 1 or 7.

In another aspect, the present disclosure provides a TAP binding to an HLA-A*30:01 molecule, comprising or consisting of the sequence of SEQ ID NO: 6.

In another aspect, the present disclosure provides a TAP binding to an HLA-A*33:01 molecule, comprising or consisting of the sequence of SEQ ID NO: 7.

In another aspect, the present disclosure provides a TAP binding to an HLA-A*33:03 molecule, comprising or consisting of the sequence of SEQ ID NO: 8.

In another aspect, the present disclosure provides a TAP binding to an HLA-A*34:02 molecule, comprising or consisting of the sequence of SEQ ID NO: 10 or 11.

In another aspect, the present disclosure provides a TAP binding to an HLA-B*07:02 molecule, comprising or consisting of the sequence of SEQ ID NO: 2, 3 or 9.

In another aspect, the present disclosure provides a TAP binding to an HLA-B*08:01 molecule, comprising or consisting of the sequence of SEQ ID NO: 2.

In another aspect, the present disclosure provides a TAP binding to an HLA-B*27:05 molecule, comprising or consisting of the sequence of SEQ ID NO: 4.

In another aspect, the present disclosure provides a TAP binding to an HLA-B*39:01 molecule, comprising or consisting of the sequence of SEQ ID NO: 2.

The TAPs of the disclosure may be produced by expression in a host cell comprising a nucleic acid encoding the TAPs (recombinant expression) or by chemical synthesis (e.g., solid-phase peptide synthesis). Peptides can be readily synthesized by manual and/or automated solid phase procedures well known in the art. Suitable syntheses can be performed for example by utilizing “T-boc” or “Fmoc” procedures. Techniques and procedures for solid phase synthesis are described in for example Solid Phase Peptide Synthesis: A Practical Approach, by E. Atherton and R. C. Sheppard, published by IRL, Oxford University Press, 1989. Alternatively, the TAPs may be prepared by way of segment condensation, as described, for example, in Liu et al., Tetrahedron Lett. 37: 933-936, 1996; Baca et al., J. Am. Chem. Soc. 117: 1881-1887, 1995; Tam et al., Int. J. Peptide Protein Res. 45: 209-216, 1995; Schnolzer and Kent, Science 256: 221-225, 1992; Liu and Tam, J. Am. Chem. Soc. 116: 4149-4153, 1994; Liu and Tam, Proc. Natl. Acad. Sci. USA 91: 6584-6588, 1994; and Yamashiro and Li, Int. J. Peptide Protein Res. 31: 322-334, 1988). Other methods useful for synthesizing the TAPs are described in Nakagawa et al., J. Am. Chem. Soc. 107: 7087-7092, 1985. In an embodiment, the TAP is chemically synthesized (synthetic peptide). Another embodiment of the present disclosure relates to a non-naturally occurring peptide wherein said peptide consists or consists essentially of an amino acid sequences defined herein and has been synthetically produced (e.g., synthesized) as a pharmaceutically acceptable salt. The salts of the TAPs according to the present disclosure differ substantially from the peptides in their state(s) in vivo, as the peptides as generated in vivo are no salts. The non-natural salt form of the peptide may modulate the solubility of the peptide, in particular in the context of pharmaceutical compositions comprising the peptides, e.g., the peptide vaccines as disclosed herein. Preferably, the salts are pharmaceutically acceptable salts of the peptides.

In an embodiment, the herein-mentioned TAP is substantially pure. A compound is “substantially pure” when it is separated from the components that naturally accompany it. Typically, a compound is substantially pure when it is at least 60%, more generally 75%, 80% or 85%, preferably over 90% and more preferably over 95%, by weight, of the total material in a sample. Thus, for example, a polypeptide that is chemically synthesized or produced by recombinant technology will generally be substantially free from its naturally associated components, e.g., components of its source macromolecule. A nucleic acid molecule is substantially pure when it is not immediately contiguous with (i.e., covalently linked to) the coding sequences with which it is normally contiguous in the naturally occurring genome of the organism from which the nucleic acid is derived. A substantially pure compound can be obtained, for example, by extraction from a natural source; by expression of a recombinant nucleic acid molecule encoding a peptide compound; or by chemical synthesis. Purity can be measured using any appropriate method such as column chromatography, gel electrophoresis, HPLC, etc. In an embodiment, the TAP is in solution. In another embodiment, the TAP is in solid form, e.g., lyophilized.

In an embodiment, the TAP is encoded by a sequence located a non-protein coding region of the genome. In an embodiment, the TAP is encoded by a sequence located in an intergenic region. In another embodiment, the TAP is encoded by a non-coding RNA (ncRNA). In another embodiment, the TAP is encoded by a sequence located in an intron.

In another aspect, the disclosure further provides a synthetic long peptide (SLP) comprising at least one of the TAP described herein. In an embodiment, the SLP comprises at least two TAPs, wherein at least one of the TAP is a TAP as described herein. In an embodiment, the SLP comprises at least 2, 3, 4, 5, 10, 15, 20, 25, 30, 35 or 40 of the TAPs described herein. In an embodiment, the SLP comprises at least one of the TAPs described herein linked to one or more amino acid sequences or domains that confer desired properties to the SLP, such as sequences or domains that stabilize the SLP and/or that improve processing and presentation by MHC molecules, for example a sequence comprising a motif cleavable by cellular proteases such as cathepsins. In another embodiment, the SLP comprises at least one of the TAPs described herein, and a TAP that binds to MHC class II molecules. The TAPs may directly attached to each other, or may be indirectly attached via a linker such as a short amino acid linker. In embodiments, the linker comprises about 4 to about 20 amino acids, or about 4 to about 15 amino acids, e.g., 4, 5, 6, 7, 8, 9, 10, 11, 12, 13, 14 or 15 amino acids. In an embodiment, the linker comprises glycine residues, serine residues, proline residues, threonine residues, or a mixture thereof. The linker may include sequences promoting the processing of the SLP to release the TAPs, such as a cathepsin-sensitive linker (e.g., a linker of 4-6 amino acids comprising the sequence LVGS, ASLG, PIVG, LLSV, VLSVG or LLSVGG, see Rabu et al., Oncoimmunology. 2019; 8(4): e1560919). In an embodiment, the SLP has a length of 500, 400, 300, 200, 150, 100, 90, 80, 70, 60 or 50 amino acids or less. In a further embodiment, the SLP has a length of 20 to 50, 45 or 40 amino acids, for example from 20 or 25 amino acids to 30, 35 or 40 amino acids.

In another aspect, the disclosure further provides a nucleic acid (isolated) encoding the herein-mentioned TAPs or a tumor antigen precursor-peptide or SLP. In an embodiment, the nucleic acid comprises from about 24 nucleotides to about 1200 nucleotides, from about 24 to about 1000, 900, 800, 700, 600, 500, 400, 300 or 200 nucleotides, for example from about 24 to about 150 or 100 nucleotides, for example 24, 27, 30, 33, 36, 39, 42, 45, 48, 51, 54, 57, 60, 53, 66, 69, or 72 nucleotides. “Isolated”, as used herein, refers to a peptide or nucleic acid molecule separated from other components that are present in the natural environment of the molecule or a naturally occurring source macromolecule (e.g., including other nucleic acids, proteins, lipids, sugars, etc.). “Synthetic”, as used herein, refers to a peptide or nucleic molecule that is not isolated from its natural sources, e.g., which is produced through recombinant technology or using chemical synthesis. In an embodiment, the nucleic acid (DNA, RNA) encoding the TAP or SLP of the disclosure comprises any one of the sequences set forth in the table below or a corresponding RNA sequence. In an embodiment, the nucleic acid encoding the TAP or SLP is an mRNA molecule. In other embodiments, the nucleic acid encoding the TAP or SLP is a self-amplifying mRNA (saRNA), a trans-amplifying mRNA (taRNA) or a circular mRNA (circRNA) (see, e.g., Liu et al., Nature Reviews Cancer, Volume 23, August 2023, pages 526-543).

TABLE 1
Nucleotide sequence of the nucleic acids encoding the TSAs identified herein

| TAP sequence (SEQ ID NO:) | Encoding nucleotide sequence (SEQ ID NO:) |
|---|---|
| SIFSEILAV (1) | TCAATATTCAGTGAGATTTTGGCAGTC (12) |
| SPFVRTQEF (2) | TCACCTTTCGTCAGGACTCAAGAGTTC (13) |
| AAPGRAVGGM (3) | GCTGCCCCAGGGAGAGCAGTGGGTGGAATG (14) |
| RTWKSVLSK (4) | AGGACATGGAAAAGTGTTTTGTCCAAA (15) |
| SLSSLVVLV (5) | TCTCTTAGTAGTTTAGTAGTTCTTGTA (16) |
| RTPGLIRVGK (6) | AGAACTCCTGGGCTCATCAGAGTTGGGAAG (17) |
| STWRLHLR (7) | TCCACCTGGAGGCTTCATCTGCGA (18) |
| EFWIRENQR (8) | GAATTCTGGATTCGGGAGAATCAGAGA (19) |
| APGSARAMI (9) | GCTCCAGGAAGTGCCAGGGCAATGATC (20) |
| EIRVDFLEK (10) | GAGATCAGGGTGGACTTTCTGGAGAAG (21) |
| KAFLTTAQK (11) | AAGGCGTTCCTAACAACTGCTCAGAAG (22) |

Of course, because of the degeneracy of the genetic code, the TAPs described herein may be encoded by variants of the above-noted sequences.

A nucleic acid of the disclosure may be used for recombinant expression of the TAP or SLP of the disclosure, and may be included in a vector or plasmid, such as a cloning vector or an expression vector, which may be transfected into a host cell. In an embodiment, the disclosure provides a cloning, expression or viral vector or plasmid comprising a nucleic acid sequence encoding the TAP of the disclosure. Alternatively, a nucleic acid encoding a TAP of the disclosure may be incorporated into the genome of the host cell. In either case, the host cell expresses the TAP or protein encoded by the nucleic acid. The term “host cell” as used herein refers not only to the particular subject cell, but to the progeny or potential progeny of such a cell. A host cell can be any prokaryotic (e.g., E. coli) or eukaryotic cell (e.g., insect cells, yeast cells, plant cells, or mammalian cells) capable of expressing the TAPs described herein. The vector or plasmid contains the necessary elements for the transcription and translation of the inserted coding sequence, and may contain other components such as resistance genes, cloning sites, etc. Methods that are well known to those skilled in the art may be used to construct expression vectors containing sequences encoding peptides or polypeptides and appropriate transcriptional and translational control/regulatory elements operably linked thereto. These methods include in vitro recombinant DNA techniques, synthetic techniques, and in vivo genetic recombination. Such techniques are described in Sambrook et al. (1989) Molecular Cloning, A Laboratory Manual, Cold Spring Harbor Press, Plainview, N.Y., and Ausubel, F. M. et al. (1989) Current Protocols in Molecular Biology, John Wiley & Sons, New York, N.Y. “Operably linked” refers to a juxtaposition of components, particularly nucleotide sequences, such that the normal function of the components can be performed. Thus, a coding sequence that is operably linked to regulatory sequences refers to a configuration of nucleotide sequences wherein the coding sequences can be expressed under the regulatory control, that is, transcriptional and/or translational control, of the regulatory sequences. “Regulatory/control region” or “regulatory/control sequence”, as used herein, refers to the non-coding nucleotide sequences that are involved in the regulation of the expression of a coding nucleic acid. Thus, the term regulatory region includes promoter sequences, regulatory protein binding sites, upstream activator sequences, and the like. The vector (e.g., expression vector) may have the necessary 5′ upstream and 3′ downstream regulatory elements such as promoter sequences such as CMV, PGK and EF-1a promoters, ribosome recognition and binding TATA box, and 3′ UTR AAUAAA transcription termination sequence for the efficient gene transcription and translation in its respective host cell. Other suitable promoters include the constitutive promoter of simian vims 40 (SV40) early promoter, mouse mammary tumor virus (MMTV), HIV LTR promoter, MoMuLV promoter, avian leukemia virus promoter, EBV immediate early promoter, and Rous sarcoma vims promoter. Human gene promoters may also be used, including, but not limited to the actin promoter, the myosin promoter, the hemoglobin promoter, and the creatine kinase promoter. In certain embodiments inducible promoters are also contemplated as part of the vectors expressing the TAP. This provides a molecular switch capable of turning on expression of the polynucleotide sequence of interest or turning off expression. Examples of inducible promoters include, but are not limited to a metallothionine promoter, a glucocorticoid promoter, a progesterone promoter, or a tetracycline promoter. Examples of vectors are plasmid, autonomously replicating sequences, and transposable elements. Additional exemplary vectors include, without limitation, plasmids, phagemids, cosmids, artificial chromosomes such as yeast artificial chromosome (YAC), bacterial artificial chromosome (BAC), or PI-derived artificial chromosome (PAC), bacteriophages such as lambda phage or M13 phage, and animal viruses. Examples of categories of animal viruses useful as vectors include, without limitation, retrovirus (including lentivirus), adenovirus, adeno-associated virus, herpesvirus (e.g., herpes simplex virus), poxvirus, baculovirus, papillomavirus, and papovavirus (e.g., SV40). Examples of expression vectors are Lenti-X™ Bicistronic Expression System (Neo) vectors (Contech), pClneo vectors (Promega) for expression in mammalian cells; pLenti4/V5-DEST™, pLenti6/V5-DEST™, and pLenti6.2/V5-GW/lacZ (Invitrogen) for lentivirus-mediated gene transfer and expression in mammalian cells. The coding sequences of the TAPs disclosed herein can be ligated into such expression vectors for the expression of the TAP in mammalian cells.

In certain embodiments, the nucleic acids encoding the TAP of the present disclosure are provided in a viral vector. A viral vector can be those derived from adenovirus, vaccinia virus, retrovirus, lentivirus, or foamy virus. As used herein, the term “viral vector” refers to a nucleic acid vector construct that includes at least one element of viral origin and has the capacity to be packaged into a viral vector particle. The viral vector can contain the coding sequence for the various TAPs or SLPs described herein in place of nonessential viral genes. In another embodiment, the nucleic acids encoding the TAP or SLP of the present disclosure are provided in a self-amplifying or self-replicating RNA (saRNA or srRNA) vectors. srRNAs are derived from positive-strand RNA viruses where the structural proteins have been removed and replaced with heterologous genes of interest. srRNAs have been successfully derived from flaviviruses, nodamura viruses, nidoviruses, and alphaviruses with therapeutic versions of the technology providing the structural proteins in trans to create single cycle viral replicon particles (VRPs) (see, e.g., Aliahmad et al. Next generation self-replicating RNA vectors for vaccines and immunotherapies. Cancer Gene Ther (2022). https://doi.org/10.1038/s41417-022-00435-8). The vector and/or particle can be utilized for the purpose of transferring DNA, RNA or other nucleic acids into cells either in vitro or in vivo. Numerous forms of viral vectors are known in the art.

In embodiment, the nucleic acid (DNA, RNA) encoding the TAP of the disclosure is comprised within a vesicle or nanoparticle such as a lipid vesicle (e.g., liposome) or lipid nanoparticle (LNP), or any other suitable vehicle (e.g., mRNA packaging systems). Thus, in another aspect, the present disclosure provides a vesicle or nanoparticle, such as a lipid vesicle or nanoparticle, comprising a nucleic acid, such as an mRNA, encoding one or more of the TAP or SLP described herein.

The term liposome as used herein in accordance with its usual meaning, referring to microscopic lipid vesicles composed of a bilayer of phospholipids or any similar amphipathic lipids (e.g., sphingolipids) encapsulating an internal aqueous medium.

The term “lipid nanoparticle” refers to liposome-like structure that may include one or more lipid bilayer rings surrounding an internal aqueous medium similar to liposomes, or micellar-like structures that encapsulates molecules (e.g., nucleic acids) in a non-aqueous core. Lipid nanoparticles typically contain cationic lipids, such as ionizable cationic lipids. Examples of cationic lipids that may be used for LNPs include DOTMA, DOSPA, DOTAP, DOPE, ePC, DLin-MC3-DMA, C12-200, ALC-0315, cKK-E12, Lipid H (SM-102), OF-Deg-Lin, A2-Iso5-2DC18, 306Oᵢ₁₀, BAME-016B, TT3, 9A1P9, FTT5, COATSOME® SS-E, COATSOME® SS-EC, COATSOME® SS-OC and COATSOME® SS-OP (see, e.g., Hou et al., Nature Reviews Materials, volume 6, pages 1078-1094 (2021); Tenchov et al., ACS Nano, 15, 16982-17015 (2021).

Liposomes and lipid nanoparticles typically include other lipid components such as lipids, lipid-like materials, and polymers that can improve liposome or nanoparticle properties, such as stability, delivery efficacy, tolerability and biodistribution. These include phospholipids (e.g., phosphatidylcholines, phosphatidylethanolamines, phosphatidylserines, and phosphatidylglycerol) such as 1,2-distearoyl-sn-glycero-3-phosphocholine (DSPC) and DOPE, sterols (such as cholesterol and cholesterol derivatives), PEGylated lipids (PEG-lipids) such as 1,2-dimyristoyl-rac-glycero-3-methoxypolyethylene glycol-2000 (PEG₂₀₀₀-DMG) and 1,2-distearoyl-rac-glycero-3-methoxypolyethylene glycol-2000 (PEG₂₀₀₀-DSG).

In an embodiment, the lipid nanoparticle according to the present disclosure comprises one or more cationic lipids, such as ionizable cationic lipids. Examples of ionizable cationic lipids include those listed in PCT publications Nos. WO 2017/061150 and WO 2019/188867, which encompassed ionizable cationic lipids commercialized under the tradenames COATSOME® SS-E, COATSOME® SS-EC, COATSOME® SS-OC and COATSOME® SS-OP.

The nucleic acid (e.g., mRNA) encoding one or more of the TAP, may be modified, for example to increase stability, improve translation efficiency, and/or reduce immunogenicity. For example, the 5′ end may be capped to stabilize the molecule and decrease immunogenicity (for example, as described in U.S. Ser. No. 10/519,189 and U.S. Ser. No. 10/494,399). One or more nucleosides of the mRNA may be modified or substituted with 1-methyl pseudo-uridine to either increase stability of the molecule or reduce recognition of the molecule by the innate immune system. A form of modified nucleosides are described in U.S. Pat. No. 9,371,511. Other types of modifications that may be made to the mRNA include incorporation of anti-reverse cap analog (ARCA), 5′-methyl-cytidine triphosphate (m5CTP), N6-methyl-adenosine-5′-triphosphate (m6ATP), 2-thio-uridine triphosphate (s2UTP), pseudouridine triphosphate, N¹Methylpseudouridine triphosphate or 5-Methoxyuridine triphosphate (5moUTP). The mRNA may also include additional modifications to the 5′- and/or 3′-untranslated regions (UTRs) and polyadenylation (polyA) tail, e.g., for improving/increasing translation (see, for example, Kim et al., Molecular & cellular toxicology vol. 18, 1 (2022): 1-8). All these modifications and other modifications to the nucleic acid (e.g., mRNA) encoding the TAP are encompassed by the present disclosure.

In another aspect, the present disclosure provides an MHC class I molecule comprising (i.e., presenting or bound to) one or more of the TAP comprising or consisting of the sequence of SEQ ID NOs: 1-11 defined herein.

In an embodiment, the MHC class I molecule is an HLA-A*02:01 molecule. In an embodiment, the MHC class I molecule is an HLA-A*03:01 molecule. In an embodiment, the MHC class I molecule is an HLA-A*26:01 molecule. In an embodiment, the MHC class I molecule is an HLA-A*30:01 molecule. In an embodiment, the MHC class I molecule is an HLA-A*33:01 molecule. In an embodiment, the MHC class I molecule is an HLA-A*33:03 molecule. In an embodiment, the MHC class I molecule is an HLA-A*34:02 molecule. In an embodiment, the MHC class I molecule is an HLA-B*07:02 molecule. In an embodiment, the MHC class I molecule is an HLA-B*8:01 molecule. In an embodiment, the MHC class I molecule is an HLA-B*27:05 molecule. In an embodiment, the MHC class I molecule is an HLA-B*39:01 molecule.

In an embodiment, the TAP (e.g., comprising or consisting of the sequence of SEQ ID NOs: 1-11 defined herein) is non-covalently bound to the MHC class I molecule (i.e., the TAP is loaded into, or non-covalently bound to the peptide binding groove/pocket of the MHC class I molecule). In another embodiment, the TAP is covalently attached/bound to the MHC class I molecule (alpha chain). In such a construct, the TAP and the MHC class I molecule (alpha chain) are produced as a synthetic fusion protein, typically with a short (e.g., 5 to 20 residues, preferably about 8-12, e.g., 10) flexible linker or spacer (e.g., a polyglycine linker). In another aspect, the disclosure provides a nucleic acid encoding a fusion protein comprising a TAP defined herein fused to an MHC class I molecule (alpha chain). In an embodiment, the MHC class I molecule (alpha chain)—peptide complex is multimerized. Accordingly, in another aspect, the present disclosure provides a multimer of MHC class I molecule loaded (covalently or not) with the herein-mentioned TAP. Such multimers may be attached to a tag, for example a fluorescent tag, which allows the detection of the multimers. A great number of strategies have been developed for the production of MHC multimers, including MHC dimers, tetramers, pentamers, octamers, etc. (reviewed in Bakker and Schumacher, Current Opinion in Immunology 2005, 17:428-433). MHC multimers are useful, for example, for the detection and purification of antigen-specific T cells. Thus, in another aspect, the present disclosure provides a method for detecting or purifying (isolating, enriching) CD8⁺ T lymphocytes specific for a TAP defined herein, the method comprising contacting a cell population with a multimer of MHC class I molecule loaded (covalently or not) with the TAP; and detecting or isolating the CD8⁺ T lymphocytes bound by the MHC class I multimers. CD8⁺ T lymphocytes bound by the MHC class I multimers may be isolated using known methods, for example fluorescence activated cell sorting (FACS) or magnetic activated cell sorting (MACS).

In yet another aspect, the present disclosure provides a cell (e.g., a host cell), in an embodiment an isolated cell, comprising the herein-mentioned nucleic acid, vector or plasmid of the disclosure, i.e., a nucleic acid or vector encoding one or more TAPs or SLPs. In another aspect, the present disclosure provides a cell expressing at its surface an MHC class I molecule (e.g., an MHC class I molecule of one of the alleles disclosed above) bound to or presenting a TAP according to the disclosure. In one embodiment, the host cell is a eukaryotic cell, such as a mammalian cell, preferably a human cell. a cell line or an immortalized cell. In another embodiment, the cell is an antigen-presenting cell (APC), such as a dendritic cell. In one embodiment, the host cell is a primary cell, a cell line or an immortalized cell. Nucleic acids and vectors can be introduced into cells via conventional transformation or transfection techniques. The terms “transformation” and “transfection” refer to techniques for introducing foreign nucleic acid into a host cell, including calcium phosphate or calcium chloride co-precipitation, DEAE-dextran-mediated transfection, lipofection, electroporation, microinjection and viral-mediated transfection. Suitable methods for transforming or transfecting host cells can for example be found in Sambrook et al. (supra), and other laboratory manuals. Methods for introducing nucleic acids into mammalian cells in vivo are also known, and may be used to deliver the vector or plasmid of the disclosure to a subject for gene therapy.

Cells such as APCs can be loaded with one or more TAPs using a variety of methods known in the art. As used herein “loading a cell” with a TAP means that RNA or DNA encoding the TAP, or the TAP, is transfected into the cells or alternatively that the APC is transformed with a nucleic acid encoding the TAP. The cell can also be loaded by contacting the cell with exogenous TAPs that can bind directly to MHC class I molecule present at the cell surface (e.g., peptide-pulsed cells). The TAPs may also be fused to a domain or motif that facilitates its presentation by MHC class I molecules, for example to an endoplasmic reticulum (ER) retrieval signal, a C-terminal Lys-Asp-Glu-Leu sequence (see Wang et al., Eur J Immunol. 2004 December; 34(12):3582-94).

In another aspect, the present disclosure provides a composition or peptide combination/pool comprising any one of, or any combination of, the TAPs defined herein (or a nucleic acid encoding said peptide(s)). In an embodiment, the composition comprises any combination of the TAPs defined herein (any combination of 2, 3, 4, 5, 6, 7, 8, 9, 10 or more TAPs), or a combination of nucleic acids encoding said TAPs). Compositions comprising any combination/sub-combination of the TAPs defined herein are encompassed by the present disclosure. In another embodiment, the combination or pool may comprise one or more known tumor antigens.

Thus, in another aspect, the present disclosure provides a composition comprising any one of, or any combination of, the TAPs defined herein (e.g., comprising or consisting of the sequence of SEQ ID NOs: 1-11 defined herein) and a cell expressing a MHC class I molecule (e.g., a MHC class I molecule of one of the alleles disclosed above). APC for use in the present disclosure are not limited to a particular type of cell and include professional APCs such as dendritic cells (DCs), Langerhans cells, macrophages and B cells, which are known to present proteinaceous antigens on their cell surface so as to be recognized by CD8⁺ T lymphocytes. For example, an APC can be obtained by inducing DCs from peripheral blood monocytes and then contacting (stimulating) the TAPs, either in vitro, ex vivo or in vivo. APC can also be activated to present a TAP in vivo where one or more of the TAPs of the disclosure are administered to a subject and APCs that present a TAP are induced in the body of the subject. The phrase “inducing an APC” or “stimulating an APC” includes contacting or loading a cell with one or more TAPs, or nucleic acids encoding the TAPs such that the TAPs are presented at its surface by MHC class I molecules. As noted herein, according to the present disclosure, the TAPs may be loaded indirectly for example using longer peptides/polypeptides comprising the sequence of the TAPs (including the native protein), which is then processed (e.g., by proteases) inside the APCs to generate the TAP/MHC class I complexes at the surface of the cells. After loading APCs with TAPs and allowing the APCs to present the TAPs, the APCs can be administered to a subject as a vaccine. For example, the ex vivo administration can include the steps of: (a) collecting APCs from a first subject, (b) contacting/loading the APCs of step (a) with a TAP to form MHC class I/TAP complexes at the surface of the APCs; and (c) administering the peptide-loaded APCs to a second subject in need for treatment.

The first subject and the second subject may be the same subject (e.g., autologous vaccine), or may be different subjects (e.g., allogeneic vaccine). Alternatively, according to the present disclosure, use of a TAP or SLP described herein (or a combination thereof) for manufacturing a composition (e.g., a pharmaceutical composition) for inducing antigen-presenting cells is provided. In addition, the present disclosure provides a method or process for manufacturing a pharmaceutical composition for inducing antigen-presenting cells, wherein the method or the process includes the step of admixing or formulating the TAP, or a combination thereof, with a pharmaceutically acceptable carrier. Cells such as APCs expressing a MHC class I molecule (e.g., any of the above-noted HLA molecules) loaded with any one of, or any combination of, the TAPs defined herein, may be used for stimulating/amplifying CD8⁺ T lymphocytes, for example autologous CD8⁺ T lymphocytes. Accordingly, in another aspect, the present disclosure provides a composition comprising any one of, or any combination of, the TAPs or SLPs defined herein (or a nucleic acid or vector encoding same); a cell expressing an MHC class I molecule and a T lymphocyte, more specifically a CD8⁺ T lymphocyte (e.g., a population of cells comprising CD8⁺ T lymphocytes).

In an embodiment, the composition further comprises a buffer, an excipient, a carrier, a diluent and/or a medium (e.g., a culture medium). In a further embodiment, the buffer, excipient, carrier, diluent and/or medium is/are pharmaceutically acceptable buffer(s), excipient(s), carrier(s), diluent(s) and/or medium (media). As used herein “pharmaceutically acceptable buffer, excipient, carrier, diluent and/or medium” includes any and all solvents, buffers, binders, lubricants, fillers, thickening agents, disintegrants, plasticizers, coatings, barrier layer formulations, lubricants, stabilizing agent, release-delaying agents, dispersion media, coatings, antibacterial and antifungal agents, isotonic agents, and the like that are physiologically compatible, do not interfere with effectiveness of the biological activity of the active ingredient(s) and that are not toxic to the subject. The use of such media and agents for pharmaceutically active substances is well known in the art (Rowe et al., Handbook of pharmaceutical excipients, 2003, 4^{th} edition, Pharmaceutical Press, London UK). Except insofar as any conventional media or agent is incompatible with the active compound (peptides, cells), use thereof in the compositions of the disclosure is contemplated. In an embodiment, the buffer, excipient, carrier and/or medium is a non-naturally occurring buffer, excipient, carrier and/or medium. In an embodiment, one or more of the TAPs defined herein, or the nucleic acids (e.g., mRNAs) encoding said one or more TAPs, are comprised within or complexed to a lipid vesicle or liposome, e.g., a cationic liposome (see, e.g., Vitor M T et al., Recent Pat Drug Deliv Formul. 2013 August; 7(2):99-110) or suitable other carriers.

In another aspect, the present disclosure provides a composition comprising one of more of the any one of, or any combination of, the TAPs or SLPs defined herein (e.g., comprising or consisting of the sequence of SEQ ID NOs: 1-11 defined herein) (or a nucleic acid such as a mRNA encoding said TAPs or SLPs, and a buffer, an excipient, a carrier, a diluent and/or a medium. For compositions comprising cells (e.g., APCs, T lymphocytes), the composition comprises a suitable medium that allows the maintenance of viable cells. Representative examples of such media include saline solution, Earl's Balanced Salt Solution (Life Technologies®) or PlasmaLyte® (Baxter International®). In an embodiment, the composition (e.g., pharmaceutical composition) is an “immunogenic composition”, “vaccine composition” or “vaccine”. The term “Immunogenic composition”, “vaccine composition” or “vaccine” as used herein refers to a composition or formulation comprising one or more TAPs, SLPs, nucleic acids or vaccine vector and which is capable of inducing an immune response against the one or more TAPs present therein when administered to a subject. Vaccination methods for inducing an immune response in a mammal (e.g., human) comprise use of a vaccine or vaccine vector to be administered by any conventional route known in the vaccine field, e.g., via a mucosal (e.g., ocular, intranasal, pulmonary, oral, gastric, intestinal, rectal, vaginal, or urinary tract) surface, via a parenteral (e.g., subcutaneous, intradermal, intramuscular, intravenous, or intraperitoneal) route, or topical administration (e.g., via a transdermal delivery system such as a patch). In an embodiment, the TAP(s) or SLP(s) (or a combination thereof) is conjugated to a carrier protein (conjugate vaccine) to increase the immunogenicity of the TAP(s) or SLP(s). The present disclosure thus provides a composition (conjugate) comprising a TAP or SLP (or a combination thereof), or a nucleic acid encoding the TAP, SLP or combination thereof, and a carrier protein. For example, the TAP(s), SLP(s) or nucleic acid(s) may be conjugated or complexed to a Toll-like receptor (TLR) ligand (see, e.g., Zom et al., Adv Immunol. 2012, 114: 177-201) or polymers/dendrimers (see, e.g., Liu et al., Biomacromolecules. 2013 Aug. 12; 14(8):2798-806) such as polymer-linked TLR agonists (see, e.g., Lynn et al., Nature Biotechnology 33: 1201-1210 (2015); Lynn et al., Nature Biotechnology 38: 320-332 (2020)). In an embodiment, the immunogenic composition or vaccine further comprises an adjuvant. “Adjuvant” refers to a substance which, when added to an immunogenic agent such as an antigen (TAPs, nucleic acids and/or cells according to the present disclosure), nonspecifically enhances or potentiates an immune response to the agent in the host upon exposure to the mixture. Examples of adjuvants currently used in the field of vaccines include (1) mineral salts (aluminum salts such as aluminum phosphate and aluminum hydroxide, calcium phosphate gels), squalene, (2) oil-based adjuvants such as oil emulsions and surfactant based formulations, e.g., MF59 (microfluidised detergent stabilised oil-in-water emulsion), QS21 (purified saponin), AS02 [SBAS2] (oil-in-water emulsion+MPL+QS-21), (3) particulate adjuvants, e.g., virosomes (unilamellar liposomal vehicles incorporating influenza haemagglutinin), AS04 ([SBAS4]aluminum salt with MPL), ISCOMS (structured complex of saponins and lipids), polylactide co-glycolide (PLG), (4) microbial derivatives (natural and synthetic), e.g., monophosphoryl lipid A (MPL), Detox (MPL+M. phlei cell wall skeleton), AGP [RC-529](synthetic acylated monosaccharide), DC_Chol (lipoidal immunostimulators able to self-organize into liposomes), OM-174 (lipid A derivative), CpG motifs (synthetic oligonucleotides containing immunostimulatory CpG motifs), modified Cholera toxin (CT) and Escherichia coli enterotoxin (LT) (genetically modified bacterial toxins to provide non-toxic adjuvant effects), (5) endogenous human immunomodulators, e.g., hGM-CSF or hIL-12 (cytokines that can be administered either as protein or plasmid encoded), Immudaptin (C3d tandem array) and/or (6) inert vehicles, such as gold particles, and the like. In an embodiment, the vaccine is an RNA vaccine.

In an embodiment, the TAP(s) (e.g., comprising or consisting of the sequence of SEQ ID NOs: 1-11) or SLPs (or a nucleic acid such as a mRNA encoding said TAP(s) or SLP(s)) or composition comprising same is/are in lyophilized form. In another embodiment, the TAP(s), SLP(s), nucleic acid(s) or composition comprising same is/are in a liquid composition. In a further embodiment, the TAP(s), SLP(s) or nucleic acid(s) is/are at a concentration of about 0.01 pg/mL to about 100 μg/mL in the composition. In further embodiments, the TAP(s) or nucleic acid(s) is/are at a concentration of about 0.2 pg/mL to about 50 μg/mL, about 0.5 pg/mL to about 10, 20, 30, 40 or 50 μg/mL, about 1 μg/mL to about 10 μg/mL, or about 2 μg/mL, in the composition.

As noted herein, cells such as APCs that express an MHC class I molecule loaded with or bound to any one of, or any combination of, the TAPs or SLPs defined herein, may be used for stimulating/amplifying CD8⁺ T lymphocytes in vivo or ex vivo. Accordingly, in another aspect, the present disclosure provides T cell receptor (TCR) molecules capable of interacting with or binding the herein-mentioned MHC class I molecule/TAP complex, and nucleic acid molecules encoding such TCR molecules, and vectors comprising such nucleic acid molecules. A TCR according to the present disclosure is capable of specifically interacting with or binding a TAP loaded on, or presented by, an MHC class I molecule, preferably at the surface of a living cell in vitro or in vivo.

The term TCR as used herein refers to an immunoglobulin superfamily member having a variable binding domain, a constant domain, a transmembrane region, and a short cytoplasmic tail; see, e.g., Janeway et al., Immunobiology: The Immune System in Health and Disease, 3rd Ed., Current Biology Publications, p. 4:33, 1997) capable of specifically binding to an antigen peptide bound to a MHC receptor. A TCR can be found on the surface of a cell and generally is comprised of a heterodimer having a and p chains (also known as TCRα and TCRβ, respectively). Like immunoglobulins, the extracellular portion of TCR chains (e.g., α-chain, β-chain) contain two immunoglobulin regions, a variable region (e.g., TCR variable a region or Vα and TCR variable β region or Vβ; typically amino acids 1 to 116 based on Rabat numbering at the N-terminus), and one constant region (e.g., TCR constant domain α or Cα and typically amino acids 117 to 259 based on Rabat, TCR constant domain β or Cβ, typically amino acids 117 to 295 based on Rabat) adjacent to the cell membrane. Also, like immunoglobulins, the variable domains contain complementary determining regions (CDRs. 3 in each chain) separated by framework regions (FRs). In certain embodiments, a TCR is found on the surface of T cells (or T lymphocytes) and associates with the CD3 complex.

A TCR and in particular nucleic acids encoding a TCR of the disclosure may for instance be applied to genetically transform/modify T lymphocytes (e.g., CD8⁺ T lymphocytes) or other types of lymphocytes generating new T lymphocyte clones that specifically recognize an MHC class I/TAP complex. In a particular embodiment, T lymphocytes (e.g., CD8⁺ T lymphocytes) obtained from a patient are transformed to express one or more TCRs that recognize a TAP and the transformed cells are administered to the patient (autologous cell transfusion). In a particular embodiment, T lymphocytes (e.g., CD8⁺ T lymphocytes) obtained from a donor are transformed to express one or more TCRs that recognize a TAP and the transformed cells are administered to a recipient (allogenic cell transfusion). In another embodiment, the disclosure provides a T lymphocyte e.g., a CD8⁺ T lymphocyte transformed/transfected by a vector or plasmid encoding a TAP-specific TCR. In a further embodiment the disclosure provides a method of treating a patient with autologous or allogenic cells transformed with a TAP-specific TCR. In certain embodiments, TCRs are expressed in primary T cells (e.g., cytotoxic T cells) by replacing an endogenous locus, e.g., an endogenous TRAC and/or TRBC locus, using, e.g., CRISPR, TALEN, zinc finger nuclease, or other targeted disruption systems.

In another embodiment, the present disclosure provides a nucleic acid encoding the above-noted TCR. In a further embodiment, the nucleic acid is present in a vector, such as the vectors described above.

In yet a further embodiment the use of a tumor antigen-specific TCR in the manufacture of autologous or allogenic cells for the treatment of leukemia, such as acute myeloid leukemia, is provided.

In some embodiments, patients treated with the compositions (e.g., pharmaceutical compositions) of the disclosure are treated prior to or following treatment with an anti-tumor agent and/or immunotherapy (e.g., CAR therapy, immune checkpoint inhibitor therapy). Compositions of the disclosure include: allogenic T lymphocytes (e.g., CD8⁺ T lymphocyte) activated ex vivo against a TAP; allogenic or autologous APC vaccines loaded with a TAP; vaccines including TAPs of nucleic acids (e.g., mRNA) encoding TAPs and allogenic or autologous T lymphocytes (e.g., CD8⁺ T lymphocyte) or lymphocytes transformed with a tumor antigen-specific TCR. The method to provide T lymphocyte clones capable of recognizing a TAP according to the disclosure may be generated for and can be specifically targeted to tumor cells expressing the TAP in a subject (e.g., graft recipient), for example an allogenic T lymphocyte and/or donor lymphocyte infusion (DLI) recipient. Hence the disclosure provides a CD8⁺ T lymphocyte encoding and expressing a T cell receptor capable of specifically recognizing or binding a TAP/MHC class I molecule complex. Said T lymphocyte (e.g., CD8⁺ T lymphocyte) may be a recombinant (engineered) or a naturally selected T lymphocyte. This specification thus provides at least two methods for producing CD8⁺ T lymphocytes of the disclosure, comprising the step of bringing undifferentiated lymphocytes into contact with a TAP/MHC class I molecule complex (typically expressed at the surface of cells, such as APCs) under conditions conducive of triggering T cell activation and expansion, which may be done in vitro or in vivo (i.e., in a patient administered with a APC vaccine wherein the APC is loaded with a TAP or in a patient treated with a TAP vaccine). Using a combination or pool of TAPs bound to MHC class I molecules, it is possible to generate a population CD8⁺ T lymphocytes capable of recognizing a plurality of TAPs. Alternatively, tumor antigen-specific or targeted T lymphocytes may be produced/generated in vitro or ex vivo by cloning one or more nucleic acids (genes) encoding a TCR (more specifically the alpha and beta chains) that specifically binds to a MHC class I molecule/TAP complex (i.e. engineered or recombinant CD8⁺ T lymphocytes). Nucleic acids encoding a TAP-specific TCR of the disclosure, may be obtained using methods known in the art from a T lymphocyte activated against a TAP ex vivo (e.g., with an APC loaded with a TAP); or from an individual exhibiting an immune response against peptide/MHC molecule complex. TAP-specific TCRs of the disclosure may be recombinantly expressed in a host cell and/or a host lymphocyte obtained from a graft recipient or graft donor, and optionally differentiated in vitro to provide cytotoxic T lymphocytes (CTLs). The nucleic acid(s) (transgene(s)) encoding the TCR alpha and beta chains may be introduced into a T cells (e.g., from a subject to be treated or another individual) using any suitable methods such as transfection (e.g., electroporation) or transduction (e.g., using viral vector). The engineered CD8⁺ T lymphocytes expressing a TCR specific for a TAP may be expanded in vitro using well known culturing methods.

The present disclosure provides methods for making the immune effector cells which express the TCRs as described herein. In one embodiment, the method comprises transfecting or transducing immune effector cells, e.g., immune effector cells isolated from a subject, such as a subject having leukemia, such that the immune effector cells express one or more TCR as described herein. In certain embodiments, the immune effector cells are isolated from an individual and genetically modified without further manipulation in vitro. Such cells can then be directly re-administered into the individual. In further embodiments, the immune effector cells are first activated and stimulated to proliferate in vitro prior to being genetically modified to express a TCR. In this regard, the immune effector cells may be cultured before or after being genetically modified (i.e., transduced or transfected to express a TCR as described herein).

Prior to in vitro manipulation or genetic modification of the immune effector cells described herein, the source of cells may be obtained from a subject. In particular, the immune effector cells for use with the TCRs as described herein comprise T cells. T cells can be obtained from a number of sources, including peripheral blood mononuclear cells (PBMCs), bone marrow, lymph nodes tissue, cord blood, thymus issue, tissue from a site of infection, ascites, pleural effusion, spleen tissue, and tumors. In certain embodiments, T cell can be obtained from a unit of blood collected from the subject using any number of techniques known to the skilled person, such as FICOLL™ separation. In one embodiment, cells from the circulating blood of an individual are obtained by apheresis. The apheresis product typically contains lymphocytes, including T cells, monocytes, granulocyte, B cells, other nucleated white blood cells, red blood cells, and platelets. In one embodiment, the cells collected by apheresis may be washed to remove the plasma fraction and to place the cells in an appropriate buffer or media for subsequent processing. In one embodiment of the invention, the cells are washed with PBS. In an alternative embodiment, the washed solution lacks calcium and may lack magnesium or may lack many if not all divalent cations. As would be appreciated by those of ordinary skill in the art, a washing step may be accomplished by methods known to those in the art, such as by using a semi-automated flow-through centrifuge. After washing, the cells may be resuspended in a variety of biocompatible buffers or other saline solution with or without buffer. In certain embodiments, the undesirable components of the apheresis sample may be removed in the cell directly resuspended culture media. In certain embodiments, T cells are isolated from peripheral blood mononuclear cells (PBMCs) by lysing the red blood cells and depleting the monocytes, for example, by centrifugation through a PERCOLL™ gradient. A specific subpopulation of T cells, such as CD28⁺, CD4⁺, CD8⁺, CD45RA⁺, and CD45RO⁺ T cells, can be further isolated by positive or negative selection techniques. For example, enrichment of a T cell population by negative selection can be accomplished with a combination of antibodies directed to surface markers unique to the negatively selected cells. One method for use herein is cell sorting and/or selection via negative magnetic immunoadherence or flow cytometry that uses a cocktail of monoclonal antibodies directed to cell surface markers present on the cells negatively selected. For example, to enrich for CD8+ cells by negative selection, a monoclonal antibody cocktail typically includes antibodies to CD14, CD20, CD11b, CD16, HLA-DR, and CD4. Flow cytometry and cell sorting may also be used to isolate cell populations of interest for use in the present disclosure. PBMC may be used directly for genetic modification with the TCRs using methods as described herein. In certain embodiments, after isolation of PBMC, T lymphocytes are further isolated and in certain embodiments, both cytotoxic and helper T lymphocytes can be sorted into naive, memory, and effector T cell subpopulations either before or after genetic modification and/or expansion.

The present disclosure provides isolated immune cells such as T lymphocytes (e.g., CD8⁺ T lymphocytes) that are specifically induced, activated and/or amplified (expanded) by a TAP (i.e., a TAP bound to MHC class I molecules expressed at the surface of cell), or a combination of TAPs. The present disclosure also provides a composition comprising CD8⁺ T lymphocytes capable of recognizing a TAP, or a combination thereof, according to the disclosure (i.e., one or more TAPs bound to MHC class I molecules) and said TAP(s). In another aspect, the present disclosure provides a cell population or cell culture (e.g., a CD8⁺ T lymphocyte population) enriched in T lymphocytes (e.g., CD8⁺ T lymphocytes) that specifically recognize one or more MHC class I molecule/TAP complex(es) as described herein. Such enriched population may be obtained by performing an ex vivo expansion of specific T lymphocytes using cells such as APCs that express MHC class I molecules loaded with (e.g., presenting) one or more of the TAPs disclosed herein. “Enriched” as used herein means that the proportion of tumor antigen-specific T lymphocytes (e.g., CD8⁺ T lymphocytes) in the population is significantly higher relative to a native population of cells, i.e., which has not been subjected to a step of ex vivo-expansion of specific T lymphocytes. In a further embodiment, the proportion of TAP-specific T lymphocytes (e.g., CD8⁺ T lymphocytes) in the cell population is at least about 0.5%, for example at least about 1%, 1.5%, 2% or 3%. In some embodiments, the proportion of TAP-specific T lymphocytes (e.g., CD8⁺ T lymphocytes) in the cell population is about 0.5 to about 10%, about 0.5 to about 8%, about 0.5 to about 5%, about 0.5 to about 4%, about 0.5 to about 3%, about 1% to about 5%, about 1% to about 4%, about 1% to about 3%, about 2% to about 5%, about 2% to about 4%, about 2% to about 3%, about 3% to about 5% or about 3% to about 4%. Such cell population or culture (e.g., a CD8⁺ T lymphocyte population) enriched in T lymphocytes (e.g., CD8⁺ T lymphocytes) that specifically recognizes one or more MHC class I molecule/peptide (TAP) complex(es) of interest may be used in tumor antigen-based cancer immunotherapy, as detailed below. In some embodiments, the population of TAP-specific T lymphocytes (e.g., CD8⁺ T lymphocytes) is further enriched, for example using affinity-based systems such as multimers of MHC class I molecule loaded (covalently or not) with the TAP(s) defined herein. Thus, the present disclosure provides a purified or isolated population of TAP-specific T lymphocytes (e.g., CD8⁺ T lymphocytes), e.g., in which the proportion of TAP-specific T lymphocytes (e.g., CD8⁺ T lymphocytes) is at least about 50%, 60%, 70%, 80%, 85%, 90%, 95%, 96%, 97%, 98%, 99% or 100%.

In another aspect, the present disclosure provides an antibody or an antigen-binding fragment thereof (e.g., a TCR mimic or TCR-like antibody), or a soluble TCR, that specifically binds to a complex comprising a TAP as described herein bound to an HLA molecule, such as the HLA molecules defined herein. The term “antibody or antigen-binding fragment thereof” as used herein refers to any type of antibody/antibody fragment including monoclonal antibodies (including full-length monoclonal antibodies), polyclonal antibodies, multispecific antibodies, humanized antibodies, CDR-grafted antibodies, chimeric antibodies and antibody fragments so long as they exhibit the desired antigenic specificity/binding activity. Antibody fragments comprise a portion of a full-length antibody, generally an antigen binding or variable region thereof. Examples of antibody fragments include Fab, Fab′, F(ab′)₂, and Fv fragments, diabodies, linear antibodies, single-chain antibody molecules (e.g., single-chain Fv, scFv), single domain antibodies (e.g., from camelids), shark NAR single domain antibodies, and multispecific antibodies formed from antibody fragments, single-chain diabodies (scDbs), bispecific T cell engagers (BiTEs), dual affinity retargeting molecules (DARTs), bivalent scFv-Fcs, and trivalent scFv-Fcs. Antibody fragments can also refer to binding moieties comprising CDRs or antigen binding domains including, but not limited to, V_{H} regions (V_{H}, V_{H}-V_{H}), anticalins, PepBodies, antibody-T-cell epitope fusions (Troybodies) or Peptibodies. In an embodiment, the antibody or antigen-binding fragment thereof is a single-chain antibody, preferably a single-chain Fv (scFv). In an embodiment, the antibody or antigen-binding fragment thereof comprises at least one constant domain, e.g., a constant domain of a light and/or heavy chain, or a fragment thereof. In a further embodiment, the antibody or antigen-binding fragment thereof comprises a Fragment crystallizable (Fc) fragment of the constant heavy chain of an antibody. In an embodiment, the antibody or antigen-binding fragment is a scFv comprising a Fc fragment (scFV-Fc). In an embodiment, the scFv component is connected to the Fc fragment by a linker, for example a hinge. The presence of an Fc region is useful to induce a complement-dependent cytotoxicity (CDC), antibody-dependent cellular phagocytosis (ADCP), or antibody-dependent cellular cytotoxicity (ADCC) response against a tumor cell.

In an embodiment, the antibody or antigen-binding fragment thereof is a multispecific antibody or an antigen-binding fragment thereof, such as a bispecific antibody or an antigen-binding fragment thereof, wherein at least one of the antigen-binding domains of the multispecific antibody or antibody fragment recognize(s) a complex comprising a TAP as described herein bound to an HLA molecule. In an embodiment, at least one of the antigen-binding domains of the multispecific antibody or antibody fragment recognize(s) an immune cell effector molecule. The term “immune cell effector molecule” refers to a molecule (e.g., protein) expressed by an immune cell and whose engagement by the multispecific antibody or antibody fragment leads to activation of the immune cells. Examples of immune cell effector molecules include the CD3 signaling complex in T cells such as CD8 T cells and the various activating receptors on NK cells (NKG2D, KIR2DS, NKp44, etc.). In a further embodiment, at least one of the antigen-binding domains of the multispecific antibody or antibody fragment recognize(s) and engage(s) the CD3 signaling complex in T cells (e.g., anti-CD3). In a further embodiment, the multispecific antibody or antibody fragment is a single-chain diabody (scDb). In a further embodiment, the scDb comprises a first antibody fragment (e.g., scFv) that binds to a complex comprising a TAP as described herein bound to an HLA molecule and a second antibody fragment (e.g., scFv) that binds to and engages an immune cell effector molecule, such as the CD3 signaling complex in T cells (e.g., anti-CD3 scFv). Such constructs may be used for example to induce the cytotoxic T cell-mediated killing of tumor cells expressing the tumor antigen/MHC complex recognized by the multispecific antibody or antibody fragment. Antibodies or antigen-binding fragments thereof may also be used as a chimeric antigen receptor (CAR) to produce CAR T cells, CAR NK cells, etc. CAR combines a ligand-binding domain (e.g., antibody or antibody fragment) that provides specificity for a desired antigen (e.g., MHC/TAP complex) with an activating intracellular domain (or signal transducing domain) portion, such as a T cell or NK cell activating domain, providing a primary activation signal. Antigen-binding fragments of antibodies, and more particularly scFv, capable of binding to molecules expressed by tumor cells are commonly used as ligand-binding domains in CAR.

In an embodiment, the soluble TCR is a soluble therapeutic bispecific TCR (see, e.g., Robinson et al., FEBS J. 2021 November; 288(21):6159-6173; Dilchert et al., Antibodies (Basel). 2022 May 10; 11(2):34).

In an embodiment, the soluble TCR (e.g., TCR-mimic), antibody or antibody fragment is attached to an antitumor agent to form an antibody-drug conjugate (ADC). Such ADC permits to target the antitumor agent to tumor cells expressing one or more of the TAPs described herein (see, e.g., Shen et al., Asian J Pharm Sci. 2020 November; 15(6):777-785).

The present disclosure also provides a nucleic acid such as an mRNA encoding the soluble TCR, antibody, antibody fragment or CAR described herein. Such nucleic acids may be formulated into suitable vehicles such as lipid nanoparticles are described above, and may be used in the treatment of cancers such as leukemia, as described below.

Thus, in another aspect, the present disclosure provides a host cell, preferably an immune cell such as a T cell or NK cell, expressing the antibody or antibody fragment (e.g., scFv) described herein.

The present disclosure further relates to a pharmaceutical composition or vaccine comprising the above-noted immune cell (CD8⁺ T lymphocytes, CAR T cell) or population of TAP-specific CD8⁺ T lymphocytes. Such pharmaceutical composition or vaccine may comprise one or more pharmaceutically acceptable excipients and/or adjuvants, as described above.

In another aspect, the present disclosure further relates to the use of any of the TAP or SLP comprising or consisting of any of the sequences of SEQ ID NO:1-11, nucleic acid, expression vector, T cell receptor, antibody/antibody fragment, cell (e.g., T lymphocyte, APC, CAR T cell), and/or composition according to the present disclosure, or any combination thereof, as a medicament or in the manufacture of a medicament for the treatment of cancer (e.g., leukemia, such as AML). The present disclosure relates to any TAP, SLP, nucleic acid, expression vector, T cell receptor, antibody/antibody fragment, cell (e.g., T lymphocyte, APC), and/or composition (e.g., vaccine composition) according to the present disclosure, or any combination thereof, for use in the treatment of cancer (e.g., leukemia, such as AML), e.g., as a leukemia vaccine. The TAP sequences identified herein may be used for the production of synthetic peptides to be used i) for in vitro priming and expansion of tumor antigen-specific T cells to be injected into tumor patients and/or ii) as vaccines to induce or boost the anti-tumor T cell response in cancer (e.g., leukemia, such as AML) patients.

In another aspect, the present disclosure provides the use of a TAP or SLP described herein (e.g., comprising or consisting of any of the sequences of SEQ ID NO:1-11), or a combination thereof (e.g., a peptide pool), or of one or more nucleic acid(s) encoding the TAP(s) or SLP(s), as a vaccine for treating cancer (e.g., leukemia, such as AML) in a subject. The present disclosure also provides the TAP or SLP described herein, or a combination thereof (e.g., a peptide pool), or of one or more nucleic acid(s) encoding the TAP(s) or SLP(s), for use as a vaccine for treating cancer (e.g., leukemia, such as AML) in a subject. In an embodiment, the subject is a recipient of TAP-specific T lymphocytes (e.g., CD8⁺ T lymphocytes). Accordingly, in another aspect, the present disclosure provides a method of treating leukemia such as AML (e.g., of reducing the number of tumor cells, killing tumor cells), said method comprising administering (infusing) to a subject in need thereof an effective amount of T lymphocytes (e.g., CD8⁺ T lymphocytes) recognizing (i.e., expressing a TCR that binds) one or more MHC class I molecule/TAP complexes (expressed at the surface of a cell such as an APC). In an embodiment, the method further comprises administering an effective amount of the TAP, SLP, or a combination thereof, or of one or more nucleic acid(s) encoding the TAP(s) or SLP(s), and/or a cell (e.g., an APC such as a dendritic cell) expressing MHC class I molecule(s) loaded with the TAP(s), to said subject after administration/infusion of said CD8⁺ T lymphocytes. In yet a further embodiment, the method comprises administering to a subject in need thereof a therapeutically effective amount of a dendritic cell loaded with one or more TAPs. In yet a further embodiment the method comprises administering to a patient in need thereof a therapeutically effective amount of an allogenic or autologous cell that expresses a recombinant TCR that binds to a TAP presented by an MHC class I molecule.

In another aspect, the present disclosure provides the use of T lymphocytes (e.g., CD8⁺ T lymphocytes) that recognize one or more MHC class I molecules loaded with (presenting) a TAP, or a combination thereof, for treating leukemia such as AML (e.g., of reducing the number of tumor cells, killing tumor cells) in a subject. In another aspect, the present disclosure provides the use of T lymphocytes (e.g., CD8⁺ T lymphocytes) that recognize one or more MHC class I molecules loaded with (presenting) a TAP or SLP, or a combination thereof, for the preparation/manufacture of a medicament for treating leukemia such as AML (e.g., for reducing the number of tumor cells, killing tumor cells), such as a lymphoblastic leukemia, in a subject. In another aspect, the present disclosure provides T lymphocytes (e.g., CD8⁺ T lymphocytes) that recognize one or more MHC class I molecule(s) loaded with (presenting) a TAP, or a combination thereof, for use in the treatment of leukemia such as AML (e.g., for reducing the number of tumor cells, killing tumor cells), in a subject. In a further embodiment, the use further comprises the use of an effective amount of a TAP or SLP (or a combination thereof), or of one or more nucleic acid(s) encoding the TAP(s), and/or of a cell (e.g., an APC) that expresses one or more MHC class I molecule(s) loaded with (presenting) a TAP, after the use of said TAP-specific T lymphocytes.

The present disclosure also provides a method of generating an immune response against tumor cells expressing human class I MHC molecules loaded with any of the TAP disclosed herein (e.g., comprising or consisting of any of the sequences of SEQ ID NO:1-11) or combination thereof in a subject, the method comprising administering cytotoxic T lymphocytes that specifically recognizes the class I MHC molecules loaded with the TAP or combination of TAPs. The present disclosure also provides the use of cytotoxic T lymphocytes that specifically recognizes class I MHC molecules loaded with any of the TAP or combination of TAPs disclosed herein for generating an immune response against tumor cells expressing the human class I MHC molecules loaded with the TAP or combination thereof.

In an embodiment, the cancer is a blood cancer, preferably leukemia such as acute lymphocytic leukemia (ALL), acute myeloid leukemia (AML), chronic myeloid leukemia (CML), hairy cell leukemia (HCL) and myelodysplastic syndromes (MDS). In an embodiment, the leukemia is AML. The AML treated by the methods and uses described herein may be of any type or subtype (e.g., low-, intermediate- or high-risk AML), for example AML with genetic abnormalities such as AML with a translocation between chromosomes 8 and 21 [t(8;21)], AML with a translocation or inversion in chromosome 16 [t(16;16) or inv(16)], AML with the PML-RARA fusion gene, AML with a translocation between chromosomes 9 and 11 [t(9;11)], AML with a translocation between chromosomes 6 and 9 [t(6:9)], AML with a translocation or inversion in chromosome 3 [t(3;3) or inv(3)], AML (megakaryoblastic) with a translocation between chromosomes 1 and 22 [t(1:22)], AML with the BCR-ABL1 (BCR-ABL) fusion gene, AML with mutated NPM1 gene, AML with biallelic mutations of the CEBPA gene, AML with mutated RUNX1 gene, AML with mutated ASX1 gene, AML with mutated IDH1 and/or IDH2 gene, AML with mutated FLT3 gene, AML with myelodysplasia-related changes, and AML related to previous chemotherapy or radiation.

In an embodiment, the methods or uses described herein further comprise determining the HLA class I alleles expressed by the patient prior to the treatment/use, and administering or using TAPs that bind to one or more of the HLA class I alleles expressed by the patient. For example, if it is determined that the patient expresses HLA-02*01 and HLA-B07*02, any combinations of (i) the TAPs of SEQ ID NO:1 and/or 5 (that bind to HLA-A02*01) and (ii) the TAPs of SEQ ID NO:2, 3 and/or 9 (that bind to HLA-B07*02) may be administered or used in the patient.

In some embodiments, patients treated with the compositions (e.g., pharmaceutical compositions) of the disclosure are treated prior to or following treatment with allogenic stem cell transplant (ASCL), allogenic lymphocyte infusion or autologous lymphocyte infusion.

In an embodiment, the TAP, SLP, nucleic acid, expression vector, T cell receptor, antibody/antibody fragment (e.g., antibody-drug conjugate (ADC)), cell (e.g., T lymphocyte, CAR T or NK cell, APC), and/or composition according to the present disclosure, or any combination thereof, may be used in combination with one or more additional active agents or therapies to treat leukemia (e.g., AML), such as chemotherapy (e.g., vinca alkaloids, agents that disrupt microtubule formation (such as colchicines and its derivatives, monomethyl auristatin E (MMAE)), anti-angiogenic agents, therapeutic antibodies, EGFR targeting agents, tyrosine kinase targeting agent (such as tyrosine kinase inhibitors), transitional metal complexes, proteasome inhibitors, antimetabolites (such as nucleoside analogs), alkylating agents, platinum-based agents, anthracycline antibiotics, topoisomerase inhibitors, macrolides, retinoids (such as all-trans retinoic acids or a derivatives thereof), geldanamycin or a derivative thereof (such as 17-AAG), inhibitors of CDK4/6, TGF-β, WNT-β-catenin, MYC or PI3K, surgery, immune checkpoint inhibitors or immunotherapeutic agents (e.g., PD-1/PD-L1 inhibitors such as anti-PD-1/PD-L1 antibodies, CTLA-4 inhibitors such as anti-CTLA-4 antibodies, B7-1/B7-2 inhibitors such as anti-B7-1/B7-2 antibodies, TIM3 inhibitors such as anti-TIM3 antibodies, BTLA inhibitors such as anti-BTLA antibodies, CD47 inhibitors such as anti-CD47 antibodies, GITR inhibitors such as anti-GITR antibodies), antibodies against tumor antigens (e.g., anti-CD19, anti-CD22 antibodies), cell-based therapies (e.g., CAR T cells, CAR NK cells), and cytokines such as IL-2, IL-7, IL-21, and IL-15. In an embodiment, the TAP, SLP, nucleic acid, expression vector, T cell receptor, cell (e.g., T lymphocyte, APC), and/or composition according to the present disclosure is administered/used in combination with an immune checkpoint inhibitor. In an embodiment, the TAP, SLP, nucleic acid, expression vector, T cell receptor, cell (e.g., T lymphocyte, APC), and/or composition according to the present disclosure is administered/used in combination with inhibitors of CDK4/6, TGF-β and/or WNT-β-catenin. Several CDK4/6 inhibitors are in clininal trials including Palbociclib (PD-0332991, Ibrance), Ribociclib (LEE-011, Kisqali), Abemaciclib (LY2835219, Verzenios), SHR6390 and Trilaciclib (G1T28). Inhibitors of TGF-β include antisense inhibitors such as AP12009 (Trabedersen) and ISTH0036, antibodies and ligand traps such as GC1008 (Fresolimumab), LY2382770, and P144, vaccines targeting the TGF-β pathway such as Belagenpumatucel-L (Lucanix™), and FANG™ or vigil (Gemogenovatucel-T), as well as small molecule inhibitors such as LY2157299 (Galunisertib) and TEW-7197. Inhibitors of the WNT-β-catenin pathway include amino acid starvators (asparaginase), GSK3 inhibitors, C2

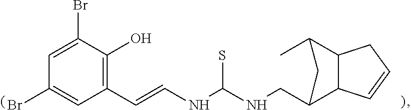 [SMILES: CC1CC(CNC(S)N/C=C/c2cc(Br)cc(Br)c2O)C2C=CCC12]

WNT974, ETC-1922159, RXC004, CGX1321, OTSA101-DTPA-90Y, Vantictumab (OMP-18R5), Ipafricept (OMP-54F28), PRI-724, SM08502, secreted frizzled-related proteins/peptides and Tankyrase inhibitors (XAV939, JW-55, RK-287107, and G007-LK). In an embodiment, the TAP, SLP, nucleic acid, expression vector, T cell receptor, antibody/antibody fragment, cell (e.g., T lymphocyte, CAR T or NK cell, APC), and/or composition according to the present disclosure, or any combination thereof according to the present disclosure is administered/used in combination one or more chemotherapeutic drugs used for the treatment of AML, or in combination with other AML therapy, for example stem cell/bone marrow transplantation.

The additional therapy may be administered prior to, concurrent with, or after the administration of the TAP, SLP, nucleic acid, expression vector, T cell receptor, antibody/antibody fragment, cell (e.g., T lymphocyte, CAR T or NK cell, APC), and/or composition according to the present disclosure.

### EXAMPLES

The present disclosure is illustrated in further details by the following non-limiting examples.

### Example 1: Identification of AML TSA Candidates

### Acquisition of Samples.

The 12 AML specimens used in this study were collected and cryopreserved at the Leukemia Cell Bank of Quebec at Maisonneuve-Rosemont Hospital, Montreal. One hundred million cells were thawed (1 min in 37° C. water bath), resuspended in 45 ml of 4° C. RPMI containing 20% FBS and washed with PBS. Two million cells were pelleted and resuspended in 1 ml Trizol® for RNA-Sequencing while the remaining 98 million were pelleted and snap-frozen in liquid nitrogen for mass spectrometry analyses.

### Immunoprecipitation of MHC I.

The W6/32 antibodies (BioXcell) were incubated in PBS for 60 min at room temperature with PureProteome protein A magnetic beads (Millipore) at a ratio of 1 mg of antibody per mL of slurry. Antibodies were covalently cross-linked to magnetic beads using dimethylpimelidate as described previously. The beads were stored at 4° C. in PBS pH 7.2 and 0.02% NaN₃. Biological replicates of cell pellets were resuspended in 1 mL PBS pH 7.2 and solubilized by adding 1 mL of detergent buffer containing PBS pH 7.2, 1% (w/v) CHAPS (Sigma) supplemented with Protease inhibitor cocktail (Sigma). After 60 min incubation with tumbling at 4° C., samples were spun at 16,000 g for 30 min at 4° C. Supernatants were transferred into new tubes containing magnetic beads coupled to W6/32 antibodies at a ratio of 10 μg of W6/32 antibody per 1×10⁶ cells. Samples were incubated with tumbling for 180 min at 4° C. and placed on a magnet to recover bound MHC I complexes to magnetic beads. Magnetic beads were first washed with 8×1 mL PBS, then with 1×1 mL of 0.1×PBS and finally with 1×1 mL of water. MHC I complexes were eluted from the magnetic beads by acidic treatment using 0.2% formic acid (FA). To remove any residual magnetic beads, eluates were transferred into 2 mL Costar mL Spin-X centrifuge tube filters (0.45 μm, Corning) and spun 2 min at 855 g. Filtrates containing peptides were separated from MHC I subunits (HLA molecules and β-2 macroglobulin) using home-made stage tips packed with twenty 1 mm diameter octadecyl (C-18) solid-phase extraction disks (EMPORE). Stage tips were pre-washed first with methanol then with 80% acetonitrile (ACN) in 0.1% trifluoroacetic acid (TFA) and finally with 0.1% FA. Samples were loaded onto the stage tips and the peptides were retained on the stage tips while the HLA molecules and β-2 macroglobulin were found in the flow through. Stage tips were washed with 0.1% FA and peptides were eluted with 30% ACN in 0.1% TFA. The peptides were dried using vacuum centrifugation and then stored at −20° C. until MS analysis.

### Liquid Chromatography-Tandem MS Analyses.

Dried peptide extracts were resuspended in 4% FA and loaded on a homemade C18 analytical column (20 cm×150 μm i.d. packed with C18 Jupiter Phenomenex) with a 106-minute gradient from 0% to 30% ACN (0.2% FA) and a 600 nL/min flow rate on an EASY-nLC II system. Samples were analyzed with an Orbitrap Exploris 480 spectrometer (Thermo Fisher Scientific) in positive ion mode with Nanoflex source at 2.8 kV. Each full MS spectrum, acquired with a 240,000 resolution was followed by 20 MS/MS spectra, where the most abundant multiply charged ions were selected for MS/MS sequencing with a resolution of 30,000, an automatic gain control target of 100%, an injection time of 700 ms, and collisional energy of 40%.

### Construction of MS Databases.

Cancer-specific proteomes were assembled using k-mer profiling as previously described (1,2). k-mers (33-nucleotide-long) present more than once in the mTECs k-mer database were removed from each cancer sample database, and the remaining k-mers were assembled into contigs. Finally, the contigs were 3-frame translated and the different polypeptides were linked with JJ linkers. This database was concatenated with corresponding sample's canonical proteome and used for TSA identification.

### MAP Identification.

LC-MS/MS data were searched against the relevant database using Peaks X Pro (Bioinformatics Solution Inc.). For peptide identification, tolerance was set at 10 ppm and 0.01 Da for precursor and fragment ions, respectively. Oxidation (M) and deamidation were set as variable modifications. Following peptide identification, the modified target-decoy approach built-in PEAKS was used to apply a sample-specific threshold on the PEAKS scores to ensure a false discovery rate (FDR) of 5%, calculated as the ratio between the number of decoy hits and the number of target hits above the score threshold. PEAKS scores corresponding to a 5% FDR for each sample were determined, and peptides that passed the threshold were further filtered to match the following criteria: peptide length between 8 and 11 amino acids, binding affinity rank to the sample's HLA alleles <2% based on NetMHCpan-4.1b (3). These filtering steps were performed with the use of MAPDP (4).

### Selection of TSAs.

AML TSA candidates were selected as previously described (1,2) based on their source RNA expression in the cancer sample of origin and mTECs (FC 210). The expression of the TSA candidates coding sequence was then evaluated in normal tissues from GTEX (n=50 samples per tissue), mTECs (n=11), purified blood and bone marrow samples (n=115), and AML samples from Leucegene cohort (5-8; n=437) using BamQuery script (2,9). TSAs listed herein are the peptides meeting the following criteria:
1. TSA source RNA is expressed at levels superior to 2-fold GTEx/mTEC samples 95th percentile expression value in at least 5% of Leucegene AML cohort samples;
2. TSA source RNA is expressed below 8.55 reads per hundred million (rphm) in more than 90% normal GTEx tissues except the testis as well as in all blood and bone marrow cells (2).
3. TSA source RNA is overexpressed in AML Leucegene cohort compared to normal myeloid progenitor cells (FC 22) (2).

### Validation of TSAs.

Prosit tool (10) was used to evaluate the correlation between predicted vs. experimental spectra of each TSA. For all TSAs with Prosit SA value below 0.7, manual inspection of the MS/MS spectra was performed. All TSAs listed herein were re-identified by a second MS search algorithm, Comet (11).

The characteristics of the TSAs identified herein are described in Tables 2A-2C.

TABLE 2A
characteristics of the TSAs identified herein

| Peptide Sequence | | | | Other alleles (promiscuous |
|---|---|---|---|---|
| (SEQ ID NO:) | Sample | Category | HLA Allele | binding alleles) |
| SIFSEILAV (1) | 17H110 | aeTSA | HLA-A*02:01 | HLA-A*26:01 |
| SPFVRTQEF (2) | 18H086 | aeTSA* | HLA-B*07:02 | HLA-A*26:01; HLA-B*08:01; |
| | | | | HLA-B*39:01 |
| AAPGRAVGGM (3) | 18H078 | aeTSA | HLA-B*07:02 | |
| RTWKSVLSK (4) | 18H086 | aeTSA | HLA-A*03:01 | HLA-B*27:05 |
| | 18H078 | | | |
| SLSSLVVLV (5) | 05H143 | aeTSA | HLA-A*02:01 | |
| RTPGLIRVGK (6) | 14H104 | aeTSA | HLA-A*30:01 | HLA-A*03:01 |
| STWRLHLR (7) | 19H066 | aeTSA | HLA-A*33:01 | |
| EFWIRENQR (8) | 18H086 | aeTSA | HLA-A*33:03 | |
| APGSARAMI (9) | 18H078 | aeTSA | HLA-B*07:02 | |
| EIRVDFLEK (10) | 05H143 | aeTSA | HLA-A*34:02 | |
| KAFLTTAQK (11) | 05H143 | aeTSA | HLA-A*34:02 | HLA-A*03:01 |

aeTSA = aberrantly expressed TSA

TABLE 2B
characteristics of the TSAs identified herein (continued)

| Peptide | Nucleotide Sequence | | | |
|---|---|---|---|---|
| SEQ ID NO: | (SEQ ID NO:) | Alignment | Strand | Biotype |
| 1 | TCAATATTCAGTGAGATT | chr13: 109724853- | − | intergenic |
| | TTGGCAGTC (12) | 109724879 | | |
| 2 | TCACCTTTCGTCAGGACT | chr7: 142708212- | + | intergenic |
| | CAAGAGTTC (13) | 142708238 | | |
| 3 | GCTGCCCCAGGGAGAGC | chr14: 22415429- | − | ncRNA_intron |
| | AGTGGGTGGAATG (14) | 22415458 | | |
| 4 | AGGACATGGAAAAGTGTT | chr10: 3052166- | + | intergenic |
| | TTGTCCAAA (15) | 3052192 | | |
| 5 | TCTCTTAGTAGTTTAGTA | chr7: 50762668- | − | intron |
| | GTTCTTGTA (16) | 50762694 | | |
| 6 | AGAACTCCTGGGCTCAT | chr1: 107407429- | − | intergenic |
| | CAGAGTTGGGAAG (17) | 107407458 | | |
| 7 | TCCACCTGGAGGCTTCAT | chr5: 43011705- | − | intergenic |
| | CTGCGA (18) | 43011728 | | |
| 8 | GAATTCTGGATTCGGGA | chr12: 116702875- | + | ncRNA_exon |
| | GAATCAGAGA (15) | 116702901 | | |
| 9 | GCTCCAGGAAGTGCCAG | chr4: 54792822- | − | intergenic |
| | GGCAATGATC (20) | 54792848 | | |
| 10 | GAGATCAGGGTGGACTT | chr10: 132496766- | − | intergenic |
| | TCTGGAGAAG (21) | 132496792 | | |
| 11 | AAGGCGTTCCTAACAACT | chr16: 81320827- | + | intron |
| | GCTCAGAAG (22) | 81320853 | | |

TABLE 2C
characteristics of the TSAs identified herein (continued)

| Peptide | | | | Overlapping ERE* |
|---|---|---|---|---|
| SEQ ID | | | | name |
| NO: | Transcript | Gene | Gene name | (class/family) |
| 1 | NA | NA | NA | |
| 2 | NA | NA | NA | MER41-int |
| | | | | (LTR/ERV1) |
| 3 | ENST00000514473 | ENSG00000251002 | AE000661.37 | |
| 4 | NA | NA | NA | |
| 5 | ENST00000335866 | ENSG00000106070 | GRB10 | antisense_L1M5 |
| | | | | (Antisense_LINE/ |
| | | | | Antisense_L1) |
| 6 | NA | NA | NA | |
| 7 | NA | NA | NA | MER4B |
| | | | | (LTR/ERV1) |
| 8 | ENST00000552718 | ENSG00000257654 | RP11- | |
| | | | 497G19.2 | |
| 9 | No Annotation | No Annotation | No | |
| | | | Annotation | |
| 10 | No Annotation | No Annotation | No | antisense_L2c |
| | | | Annotation | (Antisense_LINE/ |
| | | | | Antisense_L2) |
| 11 | ENST00000568107 | ENSG00000261609 | GAN | |

*ERE = endogenous retroviral element

Although the present invention has been described hereinabove by way of specific embodiments thereof, it can be modified, without departing from the spirit and nature of the subject invention as defined in the appended claims. In the claims, the word “comprising” is used as an open-ended term, substantially equivalent to the phrase “including, but not limited to”. The singular forms “a”, “an” and “the” include corresponding plural references unless the context clearly dictates otherwise.

### REFERENCES

1. Laumont C M, Vincent K, Hesnard L, Audemard E, Bonneil E, Laverdure J-P, et al. Noncoding regions are the main source of targetable tumor-specific antigens. Science Translational Medicine 2018; 10:eaau5516.
2. Ehx G, Larouche J-D, Durette C, Laverdure J-P, Hesnard L, Vincent K, et al. Atypical acute myeloid leukemia-specific transcripts generate shared and immunogenic MHC class-I-associated epitopes. Immunity 2021; 54:737-52.e10.
3. Reynisson B, Alvarez B, Paul S, Peters B, Nielsen M. NetMHCpan-4.1 and NetMHCllpan-4.0: improved predictions of MHC antigen presentation by concurrent motif deconvolution and integration of M S MHC eluted ligand data. Nucleic Acids Res 2020; 48:W449-W54.
4. Courcelles M, Durette C, Daouda T, Laverdure J P, Vincent K, Lemieux S, et al. MAPDP: A Cloud-Based Computational Platform for Immunopeptidomics Analyses. J Proteome Res 2020; 19:1873-81.
5. Lavallee V P, Baccelli I, Krosl J, Wilhelm B, Barabe F, Gendron P, Boucher G, Le-mieux S, Marinier A, Meloche S, et al. The transcriptomic landscape and directed chemical interrogation of MLL-rearranged acute myeloid leukemias. Nat. Genet. 2015; 47:1030-1037.
6. Lavallée V P, Lemieux S, Boucher G, Gendron P, Boivin I, Armstrong R N, Sauvageau G and Hébert J. RNA-sequencing analysis of core binding factor AML identifies recurrent ZBTB7A mutations and defines RUNX1-CBFA2T3 fusion signature. Blood 2016; 127:2498-2501.
7. Macrae T, Sargeant T, Lemieux S, Hebert J, Deneault E, Sauvageau G. RNA-Seq reveals spliceosome and proteasome genes as most consistent transcripts in human cancer cells. PLoS One. 2013 Sep. 17; 8(9):e72884.
8. Pabst C, Bergeron A, Lavallée V P, Yeh J, Gendron P, Norddahl G L, Krosl J, Boivin I, Deneault E, Simard J, Imren S, Boucher G, Eppert K, Herold T, Bohlander S K, Humphries K, Lemieux S, Hébert J, Sauvageau G, Barabé F. GPR56 identifies primary human acute myeloid leukemia cells with high repopulating potential in vivo. Blood. 2016 Apr. 21; 127(16):2018-27.
9. Ruiz Cuevas M V et al. BamQuery: a new proteogenomic tool to explore the immunopeptidome. Manuscript in preparation.
10. Gessulat, S., Schmidt, T., Zolg, D. P. et al. Prosit: proteome-wide prediction of peptide tandem mass spectra by deep learning. Nat Methods 16, 509-518 (2019).
11. Eng J K, Hoopmann M R, Jahan T A, Egertson J D, Noble W S, MacCoss M J. A deeper look into Comet-implementation and features. J Am Soc Mass Spectrom 2015; 26:1865-74.

1-59. (canceled)

60. A method of treating myelodysplastic syndrome (MDS) or leukemia in a subject comprising administering to the subject an effective amount of:
(a) a tumor antigen peptide (TAP) comprising or consisting of any one of the sequences set forth in SEQ ID NOs: 1-11 or any combination thereof, or a synthetic long peptide (SLP) comprising at least one of the sequences set forth in SEQ ID NOs: 1-11;
(b) at least one nucleic acid encoding the TAP, combination thereof or SLP defined in (a);
(c) a vesicle or particle comprising the TAP, combination thereof or SLP defined in (a) or the at least one nucleic acid defined in (b);
(d) a composition comprising the TAP, combination thereof or SLP defined in (a), the at least one nucleic acid defined in (b), or the vesicle or particle defined in (c), and a pharmaceutically acceptable carrier;
(e) a vaccine comprising the TAP, combination thereof or SLP defined in (a), the at least one nucleic acid defined in (b), the vesicle or particle defined in (c), or the composition defined in (d), and an adjuvant;
(f) a cell expressing at its surface major histocompatibility complex (MHC) class I molecules comprising the TAP or combination thereof defined in (a) in their peptide binding groove;
(g) a cell expressing at its cell surface a T-cell receptor (TCR) that specifically recognizes MHC class I molecules expressed at the surface of the cell defined in (f); or
(h) a soluble TCR, an antibody or an antigen-binding fragment thereof that specifically binds to the MHC class I molecules expressed at the surface of the cell defined in (f).

61. The method of claim 60, wherein the leukemia is a myeloid leukemia.

62. The method of claim 61, wherein said myeloid leukemia is acute myeloid leukemia (AML).

63. The method of claim 60, further comprising administering at least one additional antitumor agent or therapy to the subject.

64. The method of claim 63, wherein said at least one additional antitumor agent or therapy is a chemotherapeutic agent, immunotherapy, an immune checkpoint inhibitor, radiotherapy or surgery.

65. The method of claim 60, wherein the method comprises administering to the subject an effective amount of the at least one nucleic acid defined in (b).

66. The method of claim 65, wherein the at least one nucleic acid is at least one mRNA molecule.

67. The method of claim 66, wherein the at least mRNA molecule is encapsulated in a vesicle or particle.

68. The method of claim 67, wherein the vesicle is a lipid nanoparticle (LNP).

69. The method of claim 68, wherein the LNP comprises a cationic lipid.

70. A vaccine comprising:
(a) (i) a tumor antigen peptide (TAP) comprising or consisting of any one of the sequences set forth in SEQ ID NOs: 1-11 or any combination thereof, a synthetic long peptide (SLP) comprising at least one of the sequences set forth in SEQ ID NOs: 1-11, or (ii) at least one nucleic acid encoding the TAP, combination thereof or SLP defined in (i); and
(b) at least one vaccine adjuvant.

71. The vaccine of claim 70, comprising at least one nucleic acid encoding the TAP, combination thereof or SLP defined in (i).

72. The vaccine of claim 71, wherein the at least one nucleic acid is at least one mRNA molecule.

73. The vaccine of claim 72, wherein the at least mRNA molecule is encapsulated in a vesicle or particle.

74. The vaccine of claim 73, wherein the vesicle is a lipid nanoparticle (LNP).

75. The vaccine of claim 74, wherein the LNP comprises a cationic lipid.


SEQUENCE LISTING (22 sequences)

SEQ ID NO: 1 - protein, 9 aa, Homo sapiens
SIFSEILAV

SEQ ID NO: 2 - protein, 9 aa, Homo sapiens
SPFVRTQEF

SEQ ID NO: 3 - protein, 10 aa, Homo sapiens
AAPGRAVGGM

SEQ ID NO: 4 - protein, 9 aa, Homo sapiens
RTWKSVLSK

SEQ ID NO: 5 - protein, 9 aa, Homo sapiens
SLSSLVVLV

SEQ ID NO: 6 - protein, 10 aa, Homo sapiens
RTPGLIRVGK

SEQ ID NO: 7 - protein, 8 aa, Homo sapiens
STWRLHLR

SEQ ID NO: 8 - protein, 9 aa, Homo sapiens
EFWIRENQR

SEQ ID NO: 9 - protein, 9 aa, Homo sapiens
APGSARAMI

SEQ ID NO: 10 - protein, 9 aa, Homo sapiens
EIRVDFLEK

SEQ ID NO: 11 - protein, 9 aa, Homo sapiens
KAFLTTAQK

SEQ ID NO: 12 - DNA, 27 bp, Homo sapiens
  source 1..27: mol_type = other DNA
tcaatattcagtgagattttggcagtc

SEQ ID NO: 13 - DNA, 27 bp, Homo sapiens
  source 1..27: mol_type = other DNA
tcacctttcgtcaggactcaagagttc

SEQ ID NO: 14 - DNA, 30 bp, Homo sapiens
  source 1..30: mol_type = other DNA
gctgccccagggagagcagtgggtggaatg

SEQ ID NO: 15 - DNA, 27 bp, Homo sapiens
  source 1..27: mol_type = other DNA
aggacatggaaaagtgttttgtccaaa

SEQ ID NO: 16 - DNA, 27 bp, Homo sapiens
  source 1..27: mol_type = other DNA
tctcttagtagtttagtagttcttgta

SEQ ID NO: 17 - DNA, 30 bp, Homo sapiens
  source 1..30: mol_type = other DNA
agaactcctgggctcatcagagttgggaag

SEQ ID NO: 18 - DNA, 24 bp, Homo sapiens
  source 1..24: mol_type = other DNA
tccacctggaggcttcatctgcga

SEQ ID NO: 19 - DNA, 27 bp, Homo sapiens
  source 1..27: mol_type = other DNA
gaattctggattcgggagaatcagaga

SEQ ID NO: 20 - DNA, 27 bp, Homo sapiens
  source 1..27: mol_type = other DNA
gctccaggaagtgccagggcaatgatc

SEQ ID NO: 21 - DNA, 27 bp, Homo sapiens
  source 1..27: mol_type = other DNA
gagatcagggtggactttctggagaag

SEQ ID NO: 22 - DNA, 27 bp, Homo sapiens
  source 1..27: mol_type = other DNA
aaggcgttcctaacaactgctcagaag